(19) United States
(12) Patent Application Publication
(10) Pub. No.: US 2025/0270313 A1
(43) Pub. Date: Aug. 28, 2025

(54) CLDN18.2 ANTIBODY AND USE THEREOF

(71) Applicant: SUNSHINE LAKE PHARMA CO., LTD., Dongguan, Guangdong (CN)

(72) Inventors: Junji DONG, Dongguan (CN); Kuo ZHANG, Dongguan (CN); Tingting YU, Dongguan (CN); Xufang WANG, Dongguan (CN); Le XU, Dongguan (CN); Guanghui ZHAO, Dongguan (CN); Qunrui YE, Frederick, MD (US); Liya FENG, Dongguan (CN); Zhiheng REN, Dongguan (CN); Yan JIANG, Dongguan (CN); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 18/254,689

(22) PCT Filed: Nov. 26, 2021
(86) PCT No.: PCT/CN2021/133514

§ 371 Date: May 26, 2023

(30) Foreign Application Priority Data
Nov. 27, 2020 (CN) 202011364033.6

(51) Int. Cl. C07K 16/28; A61K 39/00; A61K 40/31; A61K 40/42; A61P 35/00; C07K 14/705; C07K 14/725; G01N 33/574; G01N 33/68

(52) U.S. Cl. CPC C07K 16/28; A61K 40/31; A61K 40/4202; A61P 35/00; C07K 14/7051; C07K 14/70517; C07K 14/70521; C07K 14/70578; G01N 33/574; G01N 33/6893; A61K 2039/505; A61K 2239/13; A61K 2239/21; A61K 2239/22; C07K 2317/33; C07K 2317/565; C07K 2317/567; C07K 2317/622; C07K 2317/76; G01N 2333/705

(57) ABSTRACT

An antibody, or an antigen-binding fragment thereof, or a chimeric antigen receptor having the ability of specifically recognizing CLDN18.2. The antibody or antigen-binding fragment thereof, or chimeric antigen receptor comprises at least one CDR sequence selected from the following or an amino acid sequence having at least 95% identity with it: the CDR sequence of light chain variable region shown in SEQ ID NO: 1, SEQ ID NO: 2, SEQ ID NO: 3, SEQ ID NO: 7, SEQ ID NO: 8 and SEQ ID NO: 9; the CDR sequence of heavy chain variable region shown in: SEQ ID NO: 4, SEQ ID NO: 5, SEQ ID NO: 6, SEQ ID NO: 10, SEQ ID NO: 11 and SEQ ID NO: 12.

### CROSS-REFERENCE TO RELATED APPLICATION

This application claims the priority and benefits of Chinese Patent Application No. 202011364033.6, filed with the State Intellectual Property Office of China on Nov. 27, 2020, which is incorporated herein by reference in its entirety.

The present application contains a Sequence Listing that has been submitted electronically in ASCII format and is hereby incorporated by reference in its entirety. The ASCII copy, created on Dec. 4, 2023, is named Substitute_Sequence_Listing_ST25.txt and is 39,632 bytes in size.

### FIELD

The present invention relates to the field of biotechnology. Specifically, the present invention relates to an CLDN18.2 antibody and use thereof. More specifically, the present invention relates to an antibody having the ability of specifically recognizing CLDN18.2 or an antigen-binding fragment thereof, a chimeric antigen receptor, an immune cell, a nucleic acid molecule, an expression vector, a recombinant cell, a pharmaceutical composition, pharmaceutical use, a kit for detecting CLDN18.2, the use of a kit for detecting CLDN18.2, and the use of screening antibodies.

### BACKGROUND

Gastric cancer ranks third in cancer-related mortality and is considered one of the most difficult cancers to be cured in the world. In patients with advanced or metastatic gastric cancer or gastroesophageal junction (GEJ) adenocarcinoma, the median overall survival (mOS) is not more than 10 months. Although human epidermal growth factor receptor 2 (HER-2) targeted therapy and immune checkpoint inhibitors have brought good news to specific populations, it is imperative to find other targets in advanced gastric cancer.

Claudins are a family of proteins whose role is to maintain tight junctions that control the exchange of molecules between cells. It is widely distributed in stomach, pancreas and lung tissues and can be used to diagnose and treat related tissue diseases. CLDN18.2 subtype is a subtype that is specifically expressed only in a small amount in stomach tissues, and not expressed in other normal tissues; it is highly selective, and is expressed in large amounts in gastric cancer cell, pancreatic cancer cell and other cancer cells. Therefore, it is an ideal target for tumor drug therapy, making CLDN18.2 specific for targeted therapy. Due to the high degree of homology between human and mouse Claudin 18.2 proteins (homology higher than 90%), conventional immunization procedures cannot produce effective immune antibodies.

It can be seen that the development of a highly specific antibody against CLDN18.2 is of great significance for the diagnosis and treatment of tumors.

### SUMMARY OF THE INVENTION

To solve the above problems, the inventors used a plasmid containing the full-length gene of hClaudin18.2 to immunize C57 mice with Claudin18.2 gene knockout, and screened two anti-Claudin18.2 murine antibodies with different affinities and binding epitopes through hybridoma fusion technology. The heavy chain and light chain variable regions of the two antibodies were obtained by gene sequencing. With the help of a linker, the light chain variable region and the heavy chain variable region were connected in series to form a VL-LINKER-VH structure for different subsequent applications.

In the first aspect of the present invention, the invention provides an antibody or antigen-binding fragment thereof having the ability of specifically recognizing CLDN 18.2. According to an embodiment of the present invention, the antibody or antigen-binding fragment thereof comprises at least one CDR sequence selected from the following or an amino acid sequence having at least 95% identity with it: the CDR sequence of light chain variable region: SEQ ID NO: 1˜3, SEQ ID NO: 7˜9; the CDR sequence of heavy chain variable region: SEQ ID NO: 4˜6, SEQ ID NO: 10˜12.

(SEQ ID NO: 1)
SQSLLNSGNQKNYL.
(SEQ ID NO: 2)
YWAST.
(SEQ ID NO: 3)
CQNDYSYPFTF.
(SEQ ID NO: 7)
SQSLFNSGNQKNYL.
(SEQ ID NO: 8)
YWAST.
(SEQ ID NO: 9)
CQNDYSYPLTF.
(SEQ ID NO: 4)
SGYTFTSYWM.
(SEQ ID NO: 5)
MIHPNSGSTN.
(SEQ ID NO: 6)
CARRYYGSISPDYW.
(SEQ ID NO: 10)
SGYTFTDYNM.
(SEQ ID NO: 11)
YINPNNGGTS.
(SEQ ID NO: 12)
CVTTRYLAVW.

According to the embodiments of the present invention, the above-mentioned antibodies can specifically target and bind to CLDN18.2 protein molecules or cells, tissues, organs, etc. with the molecules on their surfaces, thereby forming antigen-antibody complexes and exerting biological functions.

According to an embodiment of the present invention, the aforementioned antibody or antigen-binding fragment may further comprise at least one of the following additional technical features:

According to an embodiment of the present invention, the antibody comprises:
CDR1, CDR2, and CDR3 sequences of the light chain variable region shown in SEQ ID NO: 1, 2, and 3 respectively or the amino acid sequences having at least 95% identity with SEQ ID NO: 1, 2, and 3; or
CDR1, CDR2, and CDR3 sequences of the light chain variable region shown in SEQ ID NO: 7, 8 and 9 respectively or the amino acid sequences having at least 95% identity with SEQ ID NO: 7, 8 and 9.

According to an embodiment of the present invention, the antibody comprises:
CDR1, CDR2, and CDR3 sequences of the heavy chain variable region shown in SEQ ID NO: 4, 5, and 6 respectively or the amino acid sequences having at least 95% identity with SEQ ID NO: 4, 5, and 6; or
CDR1, CDR2, and CDR3 sequences of the heavy chain variable region shown in SEQ ID NO: 10, 11 and 12 respectively or amino acid sequences having at least 95% identity with SEQ ID NO: 10, 11 and 12.

According to an embodiment of the present invention, compared with peptide E, the antibody or antigen-binding fragment thereof uses peptide A as the dominant epitope that specifically recognizes CLDN 18.2, wherein the sequence of the peptide E is shown in SEQ ID NO: 14, and the sequence of the peptide A is shown in SEQ ID NO:13.

(SEQ ID NO: 14)
DLYNNPVTAVFNYQGLWRSC.
(SEQ ID NO: 13)
DQWSTQDLYNNPVTAVFNYQGC.

According to an embodiment of the present invention, the antibody with any one of the sequences of SEQ ID NO: 1˜3 and SEQ ID NO: 4˜6 only binds to the epitope where peptide A is located; the antibody with any one of the sequences of SEQ ID NO: 7-9 and SEQ ID NO: 10-12 can bind to a composite epitope composed of peptides A and E, wherein the peptide A is a dominant epitope.

According to an embodiment of the present invention, the antibody comprises at least one of a heavy chain framework region sequence and a light chain framework region sequence, wherein at least a part of at least one of the heavy chain framework region sequence and the light chain framework region sequence is derived from at least one of a murine antibody, a human antibody, a primate antibody or a mutant thereof.

According to an embodiment of the present invention, the antibody has a light chain variable region with an amino acid sequence as shown in any one of SEQ ID NO: 15 and SEQ ID NO: 16, and/or, the antibody has a heavy chain variable region with an amino acid sequence as shown in any one of SEQ ID NO: 17 and SEQ ID NO: 18.

(SEQ ID NO: 15)
DIVMTQSPSSLSVTAGEKVTMSCKSSQSLLNSGNQKNYLTWYQQKP
GQPPKLLIYWASTRESGVPDRFTGSGSGTDFTLTISSVQAEDLAVY
YCQNDYSYPFTFGSGTKLEIK.
(SEQ ID NO: 16)
DIVMTQSPSSLTVTAGEKVTMSCKSSQSLFNSGNQKNYLTWYQQKP
GQPPKLLIYWASTRESGVPDRFTGSGSGTDFTLTISSVQAEDLAVY
FCQNDYSYPLTFGAGTKLELR.
(SEQ ID NO: 17)
QVQLQQPGSELVKPGASVKLSCKASGYTFTSYWMHWVKQRPGQGLE
WIGMIHPNSGSTNYNEKFKSKATLTVDKSSSTAYMQLSSLTSEDSA
VYYCARRYYGSISPDYWGQGTTLTVSS.
(SEQ ID NO: 18)
EVQLQQSGPELVRPGASVKMSCKASGYTFTDYNMHWVKQSHGKSLE
WIGYINPNNGGTSYNQKFKGKATLTVNKSSSTAYMELRSLTSEDSA
VYYCVTTRYLAVWGTGTTVTVSS.

According to an embodiment of the present invention, the antibody has a light chain variable region with the amino acid sequence of SEQ ID NO: 15 and a heavy chain variable region with the amino acid sequence of SEQ ID NO: 17.

According to an embodiment of the present invention, the antibody has a light chain variable region with the amino acid sequence of SEQ ID NO: 16 and a heavy chain variable region with the amino acid sequence of SEQ ID NO: 18.

According to an embodiment of the present invention, the antibody comprises at least one of a heavy chain constant region and a light chain constant region, and at least a part of at least one of the heavy chain constant region and the light chain constant region is derived from at least one of a murine antibody, a human antibody, a primate antibody or a mutant thereof.

According to an embodiment of the present invention, both the light chain constant region and the heavy chain constant region of the antibody are derived from a murine IgG antibody or a mutant thereof. According to the antibody of the embodiment of the present invention, both the light chain constant region and the heavy chain constant region of the antibody are derived from human IgG4, IgG3, or IgG1.

According to an embodiment of the present invention, the antibody has a light chain with an amino acid sequence as shown in any one of SEQ ID NO: 19 and SEQ ID NO: 20:

(SEQ ID NO: 19)
DIVMTQSPSSLTVTAGEKVTMSCKSSQSLFNSGNQKNYLTWYQQKP
GQPPKLLIYWASTRESGVPDRFTGSGSGTDFTLTISSVQAEDLAVY
FCQNDYSYPLTFGAGTKLELRRADAAPTVSIFPPSSEQLTSGGASV
VCFLNNFYPKDINVKWKIDGSERQNGVLNSWTDQDSKDSTYSMSST
LTLTKDEYERHNSYTCEATHKTSTSPIVKSFNRNEC.
(SEQ ID NO: 20)
DIVMTQSPSSLSVTAGEKVTMSCKSSQSLLNSGNQKNYLTWYQQKP
GQPPKLLIYWASTRESGVPDRFTGSGSGTDFTLTISSVQAEDLAVY
YCQNDYSYPFTFGSGTKLEIKRADAAPTVSIFPPSSEQLTSGGASV
VCFLNNFYPKDINVKWKIDGSERQNGVLNSWTDQDSKDSTYSMSST
LTLTKDEYERHNSYTCEATHKTSTSPIVKSFNRNEC.

According to an embodiment of the present invention, the antibody has a heavy chain with an amino acid sequence as shown in any one of SEQ ID NO: 21 and SEQ ID NO: 22:

(SEQ ID NO: 21)
EVQLQQSGPELVRPGASVKMSCKASGYTFTDYNMHWVKQSHGKSLE
WIGYINPNNGGTSYNQKFKGKATLTVNKSSSTAYMELRSLTSEDSA
VYYCVTTRYLAVWGTGTTVTVSSAKTTPPSVYPLAPGCGDTTGSSV
TLGCLVKGYFPESVTVTWNSGSLSSSVHTFPALLQSGLYTMSSSVT
VPSSTWPSQTVTCSVAHPASSTTVDKKLEPSGPISTINPCPPCKEC
HKCPAPNLEGGPSVFIFPPNIKDVLMISLTPKVTCVVVDVSEDDPD
VQISWFVNNVEVHTAQTQTHREDYNSTIRVVSTLPIQHQDWMSGKE
FKCKVNNKDLPSPIERTISKIKGLVRAPQVYILPPPAEQLSRKDVS
LTCLVVGFNPGDISVEWTSNGHTEENYKDTAPVLDSDGSYFIYSKL
NMKTSKWEKTDSFSCNVRHEGLKNYYLKKTISRSPGK.
(SEQ ID NO: 22)
QVQLQQPGSELVKPGASVKLSCKASGYTFTSYWMHWVKQRPGQGLE
WIGMIHPNSGSTNYNEKFKSKATLTVDKSSSTAYMQLSSLTSEDSA
VYYCARRYYGSISPDYWGQGTTLTVSSAKTTPPSVYPLAPGCGDTT
GSSVTLGCLVKGYFPESVTVTWNSGSLSSSVHTFPALLQSGLYTMS
SSVTVPSSTWPSQTVTCSVAHPASSTTVDKKLEPSGPISTINPCPP
CKECHKCPAPNLEGGPSVFIFPPNIKDVLMISLTPKVTCVVVDVSE
DDPDVQISWFVNNVEVHTAQTQTHREDYNSTIRVVSTLPIQHQDWM
SGKEFKCKVNNKDLPSPIERTISKIKGLVRAPQVYILPPPAEQLSR
KDVSLTCLVVGFNPGDISVEWTSNGHTEENYKDTAPVLDSDGSYFI
YSKLNMKTSKWEKTDSFSCNVRHEGLKNYYLKKTISRSPGK.

According to an embodiment of the present invention, the antibody having the heavy chain with the amino acid sequence of SEQ ID NO: 21 and the light chain with the amino acid sequence of SEQ ID NO: 19 is the m1B6 antibody. The antibody having the heavy chain with the amino acid sequence of SEQ ID NO: 22 and the light chain with the amino acid sequence of SEQ ID NO: 20 is the m1E7 antibody.

According to an embodiment of the present invention, the antibody is a single chain antibody, a chimeric antibody, a multimeric antibody, or a CDR-grafted antibody.

According to an embodiment of the present invention, the antibody is a single chain antibody, and the single chain antibody has the amino acid sequence shown in any one of SEQ ID NO: 23 and SEQ ID NO: 24:

(SEQ ID NO: 23)
DIVMTQSPSSLTVTAGEKVTMSCKSSQSLFNSGNQKNYLTWYQQKP
GQPPKLLIYWASTRESGVPDRFTGSGSGTDFTLTISSVQAEDLAVY
FCQNDYSYPLTFGAGTKLELRGGGGSGGGGSGGGGSEVQLQQSGPE
LVRPGASVKMSCKASGYTFTDYNMHWVKQSHGKSLEWIGYINPNNG
GTSYNQKFKGKATLTVNKSSSTAYMELRSLTSEDSAVYYCVTTRYL
AVWGTGTTVTVSS.
(SEQ ID NO: 24)
DIVMTQSPSSLSVTAGEKVTMSCKSSQSLLNSGNQKNYLTWYQQKP
GQPPKLLIYWASTRESGVPDRFTGSGSGTDFTLTISSVQAEDLAVY
YCQNDYSYPFTFGSGTKLEIKGGGGSGGGGSGGGGSQVQLQQPGSE
LVKPGASVKLSCKASGYTFTSYWMHWVKQRPGQGLEWIGMIHPNSG
STNYNEKFKSKATLTVDKSSSTAYMQLSSLTSEDSAVYYCARRYYG
SISPDYWGQGTTLTVSS.

According to an embodiment of the present invention, the antibody having the amino acid sequence of SEQ ID NO: 23 is referred to as 1B6 antibody, and the antibody having the amino acid sequence of SEQ ID NO: 24 is referred to as 1E7 antibody. Among them, the antibody with the amino acid sequence shown in SEQ ID NO: 23 and SEQ ID NO: 24 can be expressed as VL-Linker-VH from N-terminus to C-terminus. VL represents the light chain variable region, and VH represents the heavy chain variable region. The linker represents the link chain connecting VL and VH.

According to an embodiment of the present invention, the antibodies may be chimeric antibodies IB6 and 1E7, and the chimeric antibody IB6 has a heavy chain of SEQ ID NO: 31 and a light chain of SEQ ID NO: 32.

(SEQ ID NO: 31)
EVQLQQSGPELVRPGASVKMSCKASGYTFTDYNMHWVKQSHGKSLE
WIGYINPNNGGTSYNQKFKGKATLTVNKSSSTAYMELRSLTSEDSA
VYYCVTTRYLAVWGTGTTVTVSSASTKGPSVFPLAPSSKSTSGGTA
ALGCLVKDYFPEPVTVSWNSGALTSGVHTFPAVLQSSGLYSLSSVV
TVPSSSLGTQTYICNVNHKPSNTKVDKKVEPKSCDKTHTCPPCPAP
ELLGGPSVFLFPPKPKDTLMISRTPEVTCVVVDVSHEDPEVKFNWY
VDGVEVHNAKTKPREEQYNSTYRVVSVLTVLHQDWLNGKEYKCKVS
NKALPAPIEKTISKAKGQPREPQVYTLPPSREEMTKNQVSLTCLVK
GFYPSDIAVEWESNGQPENNYKTTPPVLDSDGSFFLYSKLTVDKSR
WQQGNVFSCSVMHEALHNHYTQKSLSLSPGK.
(SEQ ID NO: 32)
DIVMTQSPSSLTVTAGEKVTMSCKSSQSLFNSGNQKNYLTWYQQKP
GQPPKLLIYWASTRESGVPDRFTGSGSGTDFTLTISSVQAEDLAVY
FCQNDYSYPLTFGAGTKLELRRTVAAPSVFIFPPSDEQLKSGTASV
VCLLNNFYPREAKVQWKVDNALQSGNSQESVTEQDSKDSTYSLSST
LTLSKADYEKHKVYACEVTHQGLSSPVTKSFNRGEC.

The chimeric antibody IE7 has a heavy chain of SEQ ID NO: 33 and a light chain of SEQ ID NO: 34.

(SEQ ID NO: 33)
QVQLQQPGSELVKPGASVKLSCKASGYTFTSYWMHWVKQRPGQGLE
WIGMIHPNSGSTNYNEKFKSKATLTVDKSSSTAYMQLSSLTSEDSA
VYYCARRYYGSISPDYWGQGTTLTVSSASTKGPSVFPLAPSSKSTS
GGTAALGCLVKDYFPEPVTVSWNSGALTSGVHTFPAVLQSSGLYSL
SSVVTVPSSSLGTQTYICNVNHKPSNTKVDKKVEPKSCDKTHTCPP
CPAPELLGGPSVFLFPPKPKDTLMISRTPEVTCVVVDVSHEDPEVK
FNWYVDGVEVHNAKTKPREEQYNSTYRVVSVLTVLHQDWLNGKEYK
CKVSNKALPAPIEKTISKAKGQPREPQVYTLPPSREEMTKNQVSLT
CLVKGFYPSDIAVEWESNGQPENNYKTTPPVLDSDGSFFLYSKLTV
DKSRWQQGNVFSCSVMHEALHNHYTQKSLSLSPGK.
(SEQ ID NO: 34)
DIVMTQSPSSLSVTAGEKVTMSCKSSQSLLNSGNQKNYLTWYQQKP
GQPPKLLIYWASTRESGVPDRFTGSGSGTDFTLTISSVQAEDLAVY
YCQNDYSYPFTFGSGTKLEIKRTVAAPSVFIFPPSDEQLKSGTASV
VCLLNNFYPREAKVQWKVDNALQSGNSQESVTEQDSKDSTYSLSST
LTLSKADYEKHKVYACEVTHQGLSSPVTKSFNRGEC.

According to an embodiment of the present invention, the antigen-binding fragment includes at least one of a Fab fragment, a (Fab)₂ fragment, a scFv-Fc fusion protein, a scFv-Fv fusion protein, an Fv fragment, and a minimum recognition unit.

In the second aspect of the present invention, the present invention provides a chimeric antigen receptor. According to an embodiment of the present invention, the chimeric antigen receptor comprises an extracellular region, wherein the extracellular region comprises a heavy chain variable region and a light chain variable region of a single chain antibody, and wherein the light chain variable region and the heavy chain variable region are determined according to the antibody or antigen-binding fragment thereof provided in the first aspect of the present invention. According to an embodiment of the present invention, the chimeric antigen receptor can be used for the preparation of drugs, which play a biological role based on the antigen recognition ability of antibodies.

According to an embodiment of the present invention, the chimeric antigen receptor further comprises a transmembrane region and an intracellular region, wherein the transmembrane region comprises a CD8 transmembrane region, and the intracellular region comprises an ICOS intracellular segment, 4-1BB and CD3ζ chain.

According to an embodiment of the present invention, the N-terminus of the ICOS intracellular segment is connected to the C-terminus of the CD8 transmembrane region, the C-terminus of the ICOS intracellular segment is connected to the N-terminus of the 4-1BB intracellular segment, and the C-terminus of the 4-1BB intracellular segment is connected to the N-terminus of the CD3ζ chain. The inventor found that the transmembrane region and intracellular segment of the immunostimulatory factor in the chimeric antigen receptor were connected in the above sequence, and the obtained chimeric antigen receptor had a high expression titer in the virus, Immune cells expressing the chimeric antigen receptor had a significant specific killing effect on tumor cells expressing CLDN18.2, and the non-specific killing and cytoinflammatory factor response were weak.

According to an embodiment of the present invention, the structure of the chimeric antigen receptor is: signal peptide-Anti-Claudin 18.2 scfv-CD8 hinge+CD8TM-ICOS-4-1BB-CD3ζ, wherein the amino acid sequence of the Anti-Claudin 18.2 scfv is shown in any one of SEQ ID NO: 23 and SEQ ID NO: 24.

According to an embodiment of the present invention, the amino acid sequence of the signal peptide of the chimeric antigen receptor is shown in SEQ ID NO:25.

(SEQ ID NO: 25)
MGVKVLFALICIAVAEA

According to an embodiment of the present invention, the amino acid sequence of CD8 hinge of the chimeric antigen receptor is shown in SEQ ID NO:26.

(SEQ ID NO: 26)
TTTPAPRPPTPAPTIASQPLSLRPEACRPAAGGAVHTRGLDFACD

According to an embodiment of the present invention, the amino acid sequence of CD8TM of the chimeric antigen receptor is shown in SEQ ID NO:27.

(SEQ ID NO: 27)
IYIWAPLAGTCGVLLLSLVITLYC

According to an embodiment of the present invention, the amino acid sequence of ICOS of the chimeric antigen receptor is shown in SEQ ID NO:28.

(SEQ ID NO: 28)
CWLTKKKYSSSVHDPNGEYMFMRAVNTAKKSRLTDVTL

According to an embodiment of the present invention, the amino acid sequence of 4-1BB of the chimeric antigen receptor is shown in SEQ ID NO:29.

(SEQ ID NO: 29)
KRGRKKLLYIFKQPFMRPVQTTQEEDGCSCRFPEEEEGGCEL

According to an embodiment of the present invention, the amino acid sequence of CD3ζ of the chimeric antigen receptor is shown in SEQ ID NO:30.

(SEQ ID NO: 30)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPR

In the third aspect of the present invention, the present invention provides an immune cell. According to an embodiment of the present invention, the immune cell expresses the chimeric antigen receptor proposed in the second aspect of the present invention. The immune cells according to the embodiments of the present invention have good killing effects in vivo and in vitro.

According to an embodiment of the present invention, the aforementioned nucleic acid immune cell may further include the following additional technical features:

According to an embodiment of the present invention, the immune cells include at least one of T lymphocytes, DC cells, NK cells, and NKT lymphocytes. The immune cells according to the embodiments of the present invention use the antibody provided in the first aspect of the present invention or the chimeric antigen receptor provided in the second aspect of the present invention to recognize and kill target proteins, cells, tissues, organs, etc., and then perform biological functions.

In the fourth aspect of the present invention, the present invention provides a nucleic acid molecule. According to an embodiment of the present invention, the nucleic acid molecule encodes the antibody or antigen-binding fragment thereof provided in the first aspect of the present invention or the chimeric antigen receptor provided in the second aspect of the present invention. The antibody or antigen-binding fragment encoded by the nucleic acid molecule according to the embodiment of the present invention can specifically target and bind to CLDN 18.2, and has high antigen-binding activity.

According to an embodiment of the present invention, the aforementioned nucleic acid molecule may further include the following additional technical features:

According to an embodiment of the present invention, the nucleic acid molecule is DNA.

In the fifth aspect of the present invention, the present invention provides an expression vector. According to an embodiment of the present invention, the expression vector carries the nucleic acid molecule provided in the fourth aspect of the present invention. After the expression vector according to the embodiment of the present invention is introduced into a suitable recipient cell, it can effectively realize the expression of the aforementioned antibody or antigen-binding fragment thereof that specifically recognizes CLDN18.2 under the mediation of the regulatory system, thereby realizing the mass acquisition of the antibody or antigen-binding fragment in vitro.

According to an embodiment of the present invention, the aforementioned nucleic acid molecule may further include the following additional technical features:

According to an embodiment of the present invention, the expression vector is a eukaryotic expression vector or a virus. Preferably, the virus is a lentivirus. According to an embodiment of the present invention, the eukaryotic expression vector may be a CHO cell.

In the sixth aspect of the present invention, the present invention provides a recombinant cell. According to an embodiment of the present invention, the recombinant cell carries the nucleic acid molecule provided in the fourth aspect of the present invention, or the expression vector provided in the fifth aspect of the present invention. The vector expresses the antibody or antigen-binding fragment thereof provided in the first aspect of the present invention or the chimeric antigen receptor provided in the second aspect of the present invention encoded by the nucleic acid molecule. The recombinant cells according to the embodiments of the present invention can be used for the in vitro expression and mass acquisition of the aforementioned antibodies or antigen-binding fragments specifically recognizing CLDN18.2.

According to an embodiment of the present invention, the aforementioned recombinant cell may further include at least one of the following additional technical features:

According to an embodiment of the present invention, the recombinant cell is a eukaryotic cell, and optionally, the recombinant cell is a mammalian cell. The recombinant cell according to the embodiment of the present invention is obtained by introducing the aforementioned expression vector into a host cell, and the vector can be introduced into the host cell by means of electrotransduction, liposome, injection and the like.

In the seventh aspect of the present invention, the present invention provides a pharmaceutical composition. According to an embodiment of the present invention, the pharmaceutical composition comprises the antibody or antigen-binding fragment thereof provided in the first aspect of the present invention, the chimeric antigen receptor provided in the second aspect of the present invention, the immune cell provided in the third aspect of the present invention, the nucleic acid molecule provided in the fourth aspect of the present invention, the expression vector provided in the fifth aspect of the present invention, or the recombinant cell provided in the sixth aspect of the present invention. The antibody or expressed antibody contained in the pharmaceutical composition according to the embodiment of the present invention can specifically target and bind to CLDN 18.2, has strong specificity, and exerts a good targeting effect, thereby realizing the biological effects of other drugs in the pharmaceutical composition, such as the activity inhibition of CLDN18.2 molecule, the killing of cells expressing CLDN18.2 molecule, and the like. In addition, the pharmaceutical composition according to the embodiments of the present invention can play a diagnostic role, relying on the antibody capable of specifically targeting CLDN 18.2 proposed in the first aspect of the present invention, which is combined with diagnostic reagents, and then plays a role in the diagnosis of the abnormal expression of CLDN18.2 parts of the organism, such as combining with diagnostic nuclides, nanomaterials, etc., to achieve visual observation of cells, tissues, and organs abnormally expressing CLDN18.2 in organisms, thereby assisting medical workers or scientific researchers to make more accurate judgments of the lesions.

In the eighth aspect of the present invention, the present invention provides the use of the aforementioned antibody or antigen-binding fragment thereof, the chimeric antigen receptor, the immune cell, the nucleic acid molecule, the expression vector, the recombinant cell and/or pharmaceutical composition in the manufacture of a medicament for the treatment or prevention of CLDN 18.2 related diseases. According to the application of the embodiment of the present invention, the pharmaceutical composition can be used to diagnose, treat or prevent diseases with abnormal expression of CLDN18.2, such as gastric cancer, pancreatic cancer, lung cancer and the like.

According to the embodiment of the present invention, the above-mentioned use may further include at least one of the following additional technical features:

According to an embodiment of the present invention, the CLDN 18.2 related disease includes tumors.

According to an embodiment of the present invention, the tumor includes a solid tumor expressing Claudin 18.2. Optionally, the solid tumor includes: gastric cancer, pancreatic cancer, esophageal cancer, and lung cancer.

In the eighth aspect of the present invention, the present invention provides a method of diagnosing, treating or preventing CLDN 18.2 related diseases in a subject comprising administering to the subject a therapeutically effective amount of the aforementioned antibody or antigen-binding fragment thereof, the chimeric antigen receptor, the immune cell, the nucleic acid molecule, the expression vector, the recombinant cell and/or pharmaceutical composition.

According to an embodiment of the present invention, the CLDN 18.2 related disease includes tumors.

According to an embodiment of the present invention, the tumor includes a solid tumor expressing CLDN 18.2. Optionally, the solid tumor includes: gastric cancer, pancreatic cancer, esophageal cancer, and lung cancer.

In the eighth aspect of the present invention, the present invention provides the aforementioned antibody or antigen-binding fragment thereof, the chimeric antigen receptor, the immune cell, the nucleic acid molecule, the expression vector, the recombinant cell and/or pharmaceutical composition for use in diagnosing, treating or preventing CLDN 18.2 related diseases in a subject.

According to an embodiment of the present invention, the CLDN 18.2 related disease includes tumors.

According to an embodiment of the present invention, the tumor includes a solid tumor expressing Claudin 18.2. Optionally, the solid tumor includes: gastric cancer, pancreatic cancer, esophageal cancer, and lung cancer.

In the ninth aspect of the present invention, the present invention provides a kit for detecting CLDN 18.2. According to an embodiment of the present invention, the kit includes the antibody provided in the first aspect of the present invention. The aforementioned CLDN18.2 antibody can specifically target and bind to CLDN18.2. The kit according to the embodiment of the present invention can achieve specific detection of CLDN18.2. For example, when the antibody is bound with a fluorophore, a fluorescent detection device can be used to realize the localization or real-time detection of CLDN18.2; when the antibody is bound with biotin and other markers, the qualitative or quantitative detection of CLDN18.2 can be achieved by color development reagents; the antibody can also be combined with anti-antibody to achieve a sandwich or double-sandwich method, and then achieve signal step-by-step amplification to detect CLDN 18.2.

In the tenth aspect of the present invention, the present invention provides the use of the aforementioned antibody, the aforementioned nucleic acid molecule, the aforementioned expression vector or the aforementioned recombinant cell in the manufacture of a kit for detecting CLDN 18.2 or diagnosing CLDN 18.2 related diseases. According to the embodiment of the present invention, the kit can directly detect the expression level of CLDN18.2, such as high expression, low expression, and no expression, thereby realizing the diagnosis of the disease. It can also be combined with other diagnostic reagents to obtain the status of organisms, tissues, and cells, such as combined with diagnostic nuclides, to visualize the number of cells expressing CLDN18.2 in the body, the size and location of the tissue, etc.

In the tenth aspect of the present invention, the present invention provides a method of detecting CLDN 18.2 or diagnosing CLDN 18.2 related diseases in a subject using a kit comprising the aforementioned antibody, the aforementioned nucleic acid molecule, the aforementioned expression vector or the aforementioned recombinant cell.

In the tenth aspect of the present invention, the present invention provides the aforementioned antibody, the aforementioned nucleic acid molecule, the aforementioned expression vector or the aforementioned recombinant cell for use in the preparation of a kit for detecting CLDN 18.2 or diagnosing CLDN 18.2 related diseases.

In the eleventh aspect of the present invention, the present invention provides the use of the aforementioned antibody or antigen-binding fragment thereof in screening antibody that can recognize epitopes other than peptide A in CLDN 18.2. According to the embodiment of the present invention, the antibody and the epitope of peptide A of the antigen are tightly combined to form a complex. At this time, the antibody of the present invention blocks the epitope of peptide A of the antigen, which can be used for antibody screening. The screened antibody can bind to epitopes other than peptide A of the antigen. In addition, the antibody that binds to the epitope of peptide A can also be screened, and the screened antibody that binds to the epitope of peptide A has better antigen binding ability than the antibody of the present invention.

In the eleventh aspect of the present invention, the present invention provides a method of screening antibodies comprising using the aforementioned antibody or antigen-binding fragment thereof, wherein the antibody recognizes an epitope other than peptide A in CLDN 18.2.

In the eleventh aspect of the present invention, the present invention provides the aforementioned antibody or antigen-binding fragment thereof for use in screening antibodies, wherein the antibody recognizes an epitope other than peptide A in CLDN 18.2.

The additional aspects and advantages of the present invention will be partially given in the following description, and some will become obvious from the following description, or be understood through the practice of the present invention.

### DESCRIPTION OF THR DRAWINGS

The above and/or additional aspects and advantages of the present invention will become obvious and easy to understand from the description of the embodiments in conjunction with the following drawings, in which:

FIG. 1 is a mouse serum test after immunization according to an embodiment of the present invention;

FIG. 2 is the specificity detection of m1B6/m1E7 hybridoma supernatant according to an embodiment of the present invention;

FIG. 3 is the affinity detection of m1B6/m1E7 monoclonal antibody according to an embodiment of the present invention;

FIG. 4 is the non-specificity detection of m1B6/m1E7 monoclonal antibody according to an embodiment of the present invention;

FIG. 5 is the affinity detection of anti-Claudin 18.2 scFv-Fc fusion protein according to an embodiment of the present invention;

FIG. 6 is the epitope identification of anti-Claudin 18.2 antibody according to an embodiment of the present invention;

FIG. 7 is the killing detection of different anti-Claudin 18.2 CAR-T according to an embodiment of the present invention;

FIG. 8 is the positive rate of different anti-Claudin 18.2 CAR-T according to an embodiment of the present invention;

FIG. 9 is an evaluation of an anti-Claudin 18.2 CAR-T in NCI-H460 mouse model according to an embodiment of the present invention;

FIG. 10 is an evaluation of an anti-Claudin 18.2 CAR-T in Calu-6 mouse model according to an embodiment of the present invention;

FIG. 11 is the ADCC activity detection of anti-Claudin 18.2 antibody according to an embodiment of the present invention;

FIG. 12 is the CDC activity detection of anti-Claudin 18.2 antibody according to an embodiment of the present invention;

FIG. 13 is the pharmacodynamic validation of anti-Claudin18.2 antibody in BXPC3 tumor model according to an embodiment of the present invention;

FIG. 14 is the detection of body weight change of the anti-Claudin 18.2 antibody in BXPC3 tumor model according to an embodiment of the present invention.

### EXAMPLES

The embodiments of the present invention are described in detail below. Examples of the embodiments are shown in the accompanying drawings, in which the same or similar reference numerals indicate the same or similar elements or elements with the same or similar functions. The embodiments described below with reference to the accompanying drawings are exemplary and are intended to explain the present invention and should not be construed as limiting the present invention.

In addition, the terms “first” and “second” are only used for descriptive purposes, and cannot be understood as indicating or implying relative importance or implicitly indicating the number of indicated technical features. Therefore, the features defined with “first” and “second” may explicitly or implicitly include at least one of the features. In the description of the present invention, “plurality” means at least two, such as two, three, etc., unless otherwise specifically defined.

Herein, the ADCC refers to the antibody-dependent cytotoxicity. When the IgG antibody specifically binds to the antigenic determinants on the surface of the target cell through the Fab segment, its Fc segment can bind to effector cells such as killer cells with FcγR (NK cells, monocytes-macrophages, neutrophils) to trigger the killing activity of the effector cells and directly kill the target cells.

Herein, the CDC refers to complement-dependent cytotoxicity, that is, the cytotoxicity involved in complement. The specific antibody binds to the corresponding antigen on the cell membrane surface to form a complex to activate the classical pathway of complement, and the formed membrane attack complex exerts a lytic effect on the target cell.

### Antibody

As used herein, the term “antibody” is an immunoglobulin molecule capable of binding to a specific antigen. It consists of two light chains with lighter molecular weight and two heavy chains with heavier molecular weight. The heavy (H) and light (L) chains are linked by disulfide bonds to form a tetrapeptide chain molecule. Among them, the amino acid sequence of the amino terminal (N-terminal) of the peptide chain varies greatly, which is called the variable region (V region). The carboxyl terminal (C-terminal) is relatively stable with little change, which is called the constant region (C region). The V regions of the L and H chains are referred to as VL and VH, respectively.

Some regions in the variable region have a higher degree of change in amino acid composition and arrangement order. They are called hypervariable regions (HVR). Hypervariable regions are where antigens and antibodies bind, so they are also called complementarity-determining region (CDR). There are three CDRs on both the heavy and light chain variable regions.

The present invention uses pCDNA3.4 plasmid containing the full-length gene of hClaudin18.2 to immunize CLDN 18.2 gene knockout C57 mice, and screened two anti-CLDN 18.2 murine antibodies with different affinities and binding epitopes through hybridoma fusion technology. The antibody fragment can specifically bind to the CLDN18.2 antigen, which can target the treatment of diseases that abnormally express CLDN18.2, such as tumors.

In some embodiments, the invention provides an antibody or antigen-binding fragment capable of specifically recognizing CLDN18.2, wherein the antibody or antigen-binding fragment thereof comprises at least one CDR sequence selected from the following or an amino acid sequence having at least 95% identity with it: the CDR sequences of the light chain variable region shown in SEQ ID NO: 1˜3, SEQ ID NO: 7˜9, the CDR sequences of the heavy chain variable region shown in SEQ ID NO: 4˜6, SEQ ID NO: 10˜12. In other embodiments, the antibodies or antigen-binding fragments provided by the present invention have conservative amino acid substitutions compared to the above heavy and light chains. “Antigen-binding fragment” refers to an antibody fragment that retains the ability to specifically bind to an antigen (ROR2). Examples of antigen-binding fragment include, but are not limited to, Fv fragment, disulfide bond stabilized Fv fragment (dsFv), Fab fragment, (Fab)₂, scFv-Fc fusion protein, scFv-Fv fusion protein, Fv-Fc fusion protein, at least one of a multispecific antibody, a single domain antibody, a VHH nanobody, a domain antibody, a bivalent domain antibody, or a minimal recognition unit formed from an antigen-binding fragment. “Conservative amino acid substitution” refers to the replacement of an amino acid with a residue that is biologically, chemically, or structurally similar to another amino acid. Biologically similar means that the substitution does not destroy the biological activity of the CLDN18.2 antibody or the CLDN18.2 antigen. Structural similarity refers to side chains with similar lengths of amino acids, such as alanine, glycine, or serine, or side chains of similar size. Chemical similarity means that amino acids have the same charge or are both hydrophilic or hydrophobic. For example, the hydrophobic residues isoleucine, valine, leucine or methionine are substituted with each other. Alternatively, polar amino acids can be used. For example, lysine is substituted with arginine, aspartic acid is substituted with glutamic acid, asparagine is substituted with glutamine, threonine is substituted with serine, etc.

In some embodiments, the present invention provides an antibody or antigen-binding fragment, wherein the antibody or antigen-binding fragment has a heavy chain variable region with the amino acid sequence shown in any one of SEQ ID NO: 17 and SEQ ID NO: 18 and a light chain variable region with the amino acid sequence shown in any one of SEQ ID NO: 15 and SEQ ID NO: 16. The inventors can obtain the CDR regions of the above-mentioned anti-heavy chain variable region sequence (as shown in SEQ ID NO: 4˜6, SEQ ID NO: 10˜12) and the CDR regions of the light chain variable region sequence (as shown in SEQ ID NO:1˜3, SEQ ID NO:7˜9) through the antibody sequence alignment database (NCBI, IMGT) or related software. In other embodiments, the heavy chain variable region sequence of the antibody or antigen-binding fragment has conservative amino acid substitutions compared to the amino acid sequences shown in SEQ ID NO: 17 and SEQ ID NO: 18. In some embodiments, the light chain variable region sequence of the antibody or antigen-binding fragment has conservative amino acid substitutions compared to the amino acid sequence shown in any one of SEQ ID NO: 15 and SEQ ID NO: 16. Of course, these conservative amino acid substitutions will not change the biological function of the antibody or antigen-binding fragment. In some specific ways, these conservative amino acid substitutions can occur on amino acids other than the CDR regions in the heavy chain variable region and the light chain variable region.

In the present invention, the term “m1B6” and “m1E7” can be understood as murine antibodies containing heavy and light chains, and the term “1B6” and “1E7” can be understood as single-chain antibodies formed by VH-Linker-VL. The term “chimeric antibody 1B6” and “chimeric antibody 1E7” can be understood as a chimeric antibody that retains the variable regions of murine antibodies m1B6 and m1E7, and replaces the constant regions with human IgG1.

In some preferred embodiments, the present invention provides an anti-CLDN18.2 antibody having a heavy chain with the amino acid sequence shown in any one of SEQ ID NO: 21-22, SEQ ID NO:31, SEQ ID NO:33 and a light chain with the amino acid sequence shown in any one of SEQ ID NO: 19-20, SEQ ID NO:32, SEQ ID NO:34.

In some preferred embodiments, the present invention provides an anti-CLDN18.2 single chain antibody having the amino acid sequence shown in SEQ ID NO: 23-24. The single chain antibody of the embodiment of the present invention has the structure of VL-Linker-VH from N-terminus to C-terminus. VL represents the light chain variable region, VH represents the heavy chain variable region, and Linker represents the linking chain connecting VL and VH.

In some embodiments, the anti-CLDN18.2 antibody of the present application has high ADCC activity and CDC activity with low EC50 and IC50 values, and it can effectively act on target cells.

In some embodiments, the anti-CLDN18.2 antibody of the present application can effectively inhibit tumor growth in a mouse model, and does not affect other physical indicators of the mouse, such as body weight. The antibody of the present application has a good anti-tumor effect and has less side effects.

### Immune Cell, Chimeric Antigen Receptor

The term “chimeric antigen receptor (CAR)” is a molecule that combines antibody-based specificity against a desired antigen (e.g., tumor antigen) with T cell receptor-activating intracellular domain to produce a chimeric protein that exhibits specific anti-tumor cell immune activity.

The antibody or antigen-binding fragment thereof capable of specifically recognizing CLDN18.2 can be used to prepare immune cells or chimeric antigen receptors.

To this end, the present invention also provides a chimeric antigen receptor, the chimeric antigen receptor includes an extracellular region, and the extracellular region includes the heavy chain variable region and the light chain variable region of a single chain antibody. Wherein, the light chain variable region and heavy chain variable region are selected from the aforementioned antibodies or antigen-binding fragments thereof capable of specifically recognizing CLDN 18.2. In addition to the heavy chain variable region and the light chain variable region of a single chain antibody, the extracellular region also includes a hinge region, supporting single chain variable fragments.

In some embodiments, the variable heavy and light chains in the chimeric antigen receptor are linked together by short peptides. In addition to the extracellular region, the chimeric antigen receptor further includes a transmembrane region and an intracellular region. These regions can initiate an intracellular signal cascade for antigen recognition.

In some embodiments, the intracellular region is selected from: CD3ζ, FcεRIγ, CD27, CD28, CD137, CD134, MyD88, CD40 intracellular signal region sequence, or a combination thereof; or the transmembrane region includes a CD8 or CD28 transmembrane region. In some embodiments, the chimeric antigen receptor includes an antibody, a transmembrane region and an intracellular region connected in the following order: the antibody of the present invention, CD8 and CD3ζ; the antibody of the present invention, CD8, CD137 and CD3ζ; or the antibody of the present invention, the transmembrane region of CD28 molecule, the intracellular signal region of CD28 molecule and CD3ζ; or the antibody of the present invention, the transmembrane region of CD28 molecule, the intracellular signal region of CD28 molecule, CD137 and CD3ζ.

In some embodiments, the transmembrane region includes the immunocostimulatory factor transmembrane region. In some embodiments, the immunecostimulatory factor transmembrane region may further be a CD8 transmembrane region or an ICOS transmembrane region.

In some embodiments, the intracellular region includes the intracellular segment of the immunostimulatory factor and the CD3ζ chain.

In some embodiments, the intracellular segment of the immunostimulatory factor further comprises ICOS or 4-IBB or OX-40 fused with an intracellular signaling domain derived from a CD3ζ sequence.

In some embodiments, the chimeric antigen receptor further comprises two co-stimulatory molecules fused with the CD3ζ inner domain on a single chain single vector (such as a retroviral vector) or a double chain single vector (such as a retroviral vector). The cleaved double chain single vector expresses two chains, one of which contains the scFv fused with a costimulatory molecule and the CD3ζ inner domain.

In some embodiments, the chimeric antigen receptor further comprises a cytokine receptor and a chemotactic receptor.

Based on the fact that the above-mentioned chimeric antigen receptor can prepare immune cells, the immune cell can express the above-mentioned chimeric antigen receptor.

In some embodiments, the immune cell includes at least one of T lymphocyte, DC cell, NK cell, and NKT lymphocyte. In some embodiments, the immune cell can specifically kill cancer cells with CLDN 18.2 on the surface, and has good killing effects in vivo and in vitro.

T cell expressing CAR is called CAR T cell or CAR modified T cell.

The CAR (including its functional parts and functional variants) of the embodiments of the present invention can be obtained by methods known in the art. CAR can be prepared by any suitable method for preparing polypeptides or proteins. Suitable methods for de novo synthesis of polypeptides and proteins are described in references, such as Chan et al., Fmoc Solid Phase Peptide Synthesis, Oxford University Press, Oxford, United Kingdom, 2000; Peptide and Protein Drug Analysis, Reid, R. editor, Marcel Dekker Inc., 2000; Epitope Mapping, Westwood et al. editors, Oxford University Press, Oxford, United Kingdom, 2001; and U.S. Pat. No. 5,449,752. In addition, polypeptides and proteins can be produced recombinantly using the nucleic acids described herein using standard recombinant methods. See, for example, Sambrook et al., Molecular Cloning: A Laboratory Manual, 3rd edition, Cold Spring Harbor Press, Cold Spring Harbor, NY 2001; and Ausubel et al., Current Protocols in Molecular Biology, Greene Publishing Associates and John Wiley & Sons, NY, 1994. In addition, some CARs of the present invention (including functional parts and functional variants) can be isolated and/or purified from sources such as plants, bacteria, insects, mammals such as rats, humans, and the like. Separation and purification methods are well known in the art. Alternatively, the CAR described herein (including its functional parts and functional variants) can be commercially synthesized by companies such as Synpep (Dublin, CA), Peptide Technologies Corp. (Gaithersburg, MD) and Multiple Peptide Systems (San Diego, CA). In this regard, the CAR of the present invention can be synthesized, recombined, isolated, and/or purified.

In some embodiments, the immune cell also carries the coding sequence of an exogenous cytokine; or it also expresses another chimeric antigen receptor, which does not contain CD3ζ, but contains the intracellular signal domain of CD28, the intracellular signal domain of CD137, or a combination of the two; or it also expresses a chemokine receptor; preferably, the chemokine receptor includes: CCR; or it also expresses siRNA that can reduce PD-1 expression or a protein that blocks PD-L1; or endogenous PD-1 in the cell is knocked out by gene editing technology; or it also expresses a safety switch.

In another aspect of the present invention, the present invention also provides a multifunctional immunoconjugate comprising the antibody of the present invention and a functional molecule linked to it; the functional molecule is selected from: molecule targeting tumor surface marker, molecule inhibiting tumor, molecule targeting the surface marker of immune cell or detectable marker. In some embodiments, the molecule that targets the surface marker of immune cell is an antibody that binds to the surface marker of T cells, which forms a bifunctional antibody involving T cell participation with the antibody of the present invention.

The term “costimulatory molecule” as used herein refers to a homologous binding partner on immune cells such as T cells, which specifically binds to a costimulatory ligand, thereby mediating a costimulatory response, such as but not limited to proliferation. Costimulatory molecule is cell surface molecule other than antigen receptor or their ligand, which promotes effective immune responses. Costimulatory molecule includes but is not limited to MHC I molecule, BTLA and Toll ligand receptor, as well as OX40, CD27, CD28, CDS, ICAM-1, LFA-1 (CD11a/CD18), ICOS (CD278) and 4-1BB (CD137). Examples of costimulatory molecule include, but are not limited to: CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19.

The term “scFv” refers to a fusion protein comprising at least one variable region antibody fragment including a light chain and at least one variable region antibody fragment including a heavy chain, wherein the light chain and the heavy chain variable regions are contiguous (e.g., via a synthetic linker such as a short flexible polypeptide linker) and can be expressed as a single chain polypeptide, and the scFv retains the specificity of the intact antibody from which it is derived. Unless specified, as used herein, the scFv may have the VL and VH variable regions in any order (for example, relative to the N-terminus and C-terminus of the polypeptide), and the scFv may include VL-linker-VH or may include VH-linker-VL.

The term “epitope” and other grammatical forms as used herein can refer to a part of an antigen that can be recognized by antibodies, B cells, T cells, or engineered cells. For example, the epitope can be a tumor epitope or a pathogen epitope recognized by TCR, or it can recognize multiple epitopes in an antigen. Epitopes can also be mutated.

### Nucleic Acid Molecule, Expression Vector, Recombinant Cell

In the process of preparing or obtaining these antibodies or chimeric antigen receptors, nucleic acid molecules expressing these antibodies or chimeric antigen receptors can be linked to different vectors and expressed in different cells to obtain corresponding antibodies or chimeric antigen receptors.

To this end, the present invention also provides an isolated nucleic acid molecule that encodes the aforementioned antibody or antigen-binding fragment thereof or chimeric antigen receptor.

In some preferred embodiments, the nucleic acid molecule is species-optimized for easier expression in mammalian cells.

The present invention also provides an expression vector, which comprises the aforementioned isolated nucleic acid molecule. When the isolated polynucleotide is connected to the vector, the polynucleotide can be directly or indirectly connected to the control elements on the vector, as long as these control elements can control the translation and expression of the polynucleotide. Of course, these control elements can come directly from the carrier itself, or exogenous, that is, not from the carrier itself. Of course, the polynucleotide can be operably linked to the control element. Here, “operably linked” refers to the connection of the exogenous gene to the vector, so that the control elements in the vector, such as transcription control sequence and translation control sequence, etc., can perform its expected function of regulating the transcription and translation of the exogenous gene. Of course, the polynucleotides used to encode the heavy chain and light chain of an antibody can be inserted into different vectors independently, and it is common to insert into the same vector. Commonly used vectors can be, for example, plasmids, bacteriophages, and the like.

The present invention also provides a recombinant cell, which contains the expression vector. The expression vector can be introduced into mammalian cells to construct recombinant cells, and then use these recombinant cells to express the antibodies or antigen-binding fragments provided by the present invention. By culturing the recombinant cells, the corresponding antibodies can be obtained. These usable mammalian cells may be, for example, CHO cells.

### Pharmaceutical Composition, Kit and Pharmaceutical Use and Use in the Preparation of Kit

The present invention also provides a pharmaceutical composition, which comprises the above-mentioned antibody or antigen-binding fragment thereof and a pharmaceutically acceptable carrier, and may also comprise the above-mentioned chimeric antigen receptor, immune cell, nucleic acid molecule, expression vector, recombinant cell.

The CLDN18.2 antibody provided herein can be incorporated into a pharmaceutical composition suitable for administration to a subject. Generally, these pharmaceutical compositions include the CLDN18.2 antibody provided herein.

In some embodiments, these pharmaceutical compositions further include a pharmaceutically acceptable carrier, including any solvents, solid excipients, diluents, binders, disintegrants, or other liquid excipients, dispersing agents, flavoring or suspending agents, surfactants, isotonic agents, thickeners, emulsifiers, preservatives, solid binders, glidants or lubricants, etc., which are suitable for specific target dosage forms. Except insofar as any conventional excipients incompatible with the compounds disclosed herein, such as by producing any undesirable biological effect or otherwise interacting in a deleterious manner with any other components of the pharmaceutically acceptable composition, its use is contemplated to be within the scope of this invention.

For example, the antibodies of the present invention can be incorporated into pharmaceutical compositions suitable for parenteral administration (e.g., intravenous, subcutaneous, intraperitoneal, intramuscular). These pharmaceutical compositions can be prepared in various forms. For example, liquid, semi-solid and solid dosage forms, including but not limited to liquid solutions (for example, injection solutions and infusion solutions), dispersions or suspensions, tablets, pills, powders, liposomes, and suppositories. Typical pharmaceutical compositions are in the form of injection solutions or infusion solutions. The antibody can be administered by intravenous infusion or injection, or intramuscular or subcutaneous injection.

Of course, the CLDN18.2 antibody herein can also be made into a part of a kit or other diagnostic reagents as needed. According to an embodiment of the present invention, the present invention also provides a kit, which includes the above-mentioned CLDN18.2 antibody. The kit provided by the present invention can be used, for example, for immunoblotting, immunoprecipitation, etc., which involve the use of the specific binding properties of CLDN18.2 antigen and antibody to detect. These kits may contain any one or more of the following: antagonist, CLDN18.2 antibody or drug reference material; protein purification column; immunoglobulin affinity purification buffer; cell assay diluent; instructions or literature, etc. The CLDN18.2 antibody can be used in different types of diagnostic tests, for example, it can detect the presence of various diseases or drugs, toxins or other proteins in vitro or in vivo. For example, the subject's serum or blood can be tested for related diseases. Such as cancers or tumors, these cancers or tumors can be any unregulated cell growth.

In some embodiments, the CLDN18.2 antibody can be used in combination with any detection reagent or therapeutic agent, for example, in combination with diagnostic nuclides, nanomaterials, etc., to detect the target site through the radioactivity of the nuclide, so as to obtain information about the target site; it can also be used in combination with therapeutic nuclides to use the radioactivity of the nuclides to specifically kill target cells, tissues, etc.

When using the CLDN18.2 antibody provided by the present invention to diagnose or treat or prevent the above-mentioned diseases, the CLDN18.2 antibody provided by the present invention can be provided to the subject. To this end, the present invention provides a method for treating the above-mentioned diseases, which comprises administering the antibody or antigen-binding fragment thereof provided by the present invention to a subject in need.

The terms “treatment” and “prevention” as used herein, and words derived therefrom, do not necessarily imply 100% or complete treatment or prevention. On the contrary, there are different degrees of treatment or prevention, and those of ordinary skill in the art believe that the treatment or prevention has potential benefits or therapeutic effects. In this regard, the method of the present invention can provide any amount of any level of treatment or prevention of cancer in a mammal. Moreover, the treatment or prevention provided by the method of the present invention may include the treatment or prevention of the disease being treated or prevented, such as the treatment or prevention of one or more diseases or symptoms of cancer. In addition, for the purposes of this document, “prevention” can encompass delaying the onset of a disease or its symptoms or patients.

The present invention will be described below with reference to specific embodiments. It should be noted that these embodiments are only descriptive and do not limit the present invention in any way. If the specific technology or conditions are not indicated in the examples, the technology or conditions described in the literature in the art or the product descriptions are performed. If the reagents or instruments used are not specified by the manufacturers, they are all conventional products that are commercially available.

### Example 1 The Monoclonal Screening and Activity Identification of Anti-Claudin 18.2

### 1.1 Screening of Anti-Claudin18.2 Monoclonal Antibody:

### Screening of Mouse Immune and Hybridoma:

The C57 mice with Claudin18.2 gene knockout were immunized with the pCDNA3.4 plasmid containing the full-length gene of hClaudin18.2. Each mouse was immunized with 60 μg plasmid by intramuscular injection, and a total of 10 mice were immunized. The immunization interval was 2 weeks. Blood was collected on the 7th day after 3 times of plasmid immunizations, and the serum was diluted 100 times. 293T cells with high expression of hClaudin18.2 were used to detect the immune response of mice. The mice with obvious immune response were selected for tail vein or intraperitoneal impulse immunization by using 293T cells with high expression of hClaudin18.2 (FIG. 1), and the inoculation amount of each mouse was 1E+07 cells. After 3-4 days, the mouse spleens were taken, ground with a 70 μm mesh, then fused and plated with SP2/0 cells by PEG, and CHO cells with high expression of hClaudin18.2 were used for hybridoma screening.

1.2 Preparation of Cell Lines with High Expression of hClaudin18.2 and hClaudin18.1-CHO:

The cell lines with high expression of hClaudin18.2-CHO and hClaudin18.1-CHO were collected respectively, each with about 5E+06 cells, and the cell viability was more than 95%. The cells were collected by centrifugation at 500 g for 3 min, washed with an equal volume of pre-cooled PBS containing 1% BSA and centrifuged 3 times, then resuspended in pre-cooled PBS containing 1% BSA at a density of 1E+07 cells/mL. Each type of cell was divided into 4 flow detection tubes in the amount of 100 μL per tube.

1.3 m1B6/m1E7 Anti-Claudin 18.2 Monoclonal Hybridoma Supernatant Processing:

The two aliquoted cells were numbered as hClaudin18.2-CHO-NC, hClaudin18.2-CHO-mouse secondary antibody, hClaudin18.2-CHO-m1B6, hClaudin18.2-CHO-m1E7; 100 μL of pre-cooled PBS containing 1% BSA was added to the sample numbered by NC and mouse secondary antibody and mixed well; 100 μL of the corresponding m1B6/m1E7 monoclonal hybridoma cell line supernatant was added to the flow tube corresponding to the m1B6/m1E7 label and mixed well. After all samples were reacted at 4° C. for 30 minutes, the cells were collected by centrifugation at 500 g for 3 minutes, washed with an equal volume of pre-cooled PBS containing 1% BSA, and centrifuged for 3 times, and then the cells were collected for later use.

Mouse secondary antibody dilution preparation: the PE-labeled GAM-IgG-PE-labeled (ab97024) was diluted with pre-cooled PBS containing 1% BSA at a ratio of 1:500, with a total of 2 mL. The mixture was mixed thoroughly and stored at 4° C. for later use. The diluted mouse secondary antibody diluent hClaudin18.2-CHO-mouse secondary antibody, hClaudin18.2-CHO-m1B6, hClaudin18.2-CHO-m1E7 were taken according to the amount of 200 μL per tube. The cells treated with hClaudin18.1-CHO-mouse secondary antibody, hClaudin18.1-CHO-m1B6, and hClaudin18.1-CHO-m1E7 were resuspended. After hClaudin18.2-CHO-NC and hClaudin18.1-CHO-NC were added with 200 μL of pre-cooled PBS containing 1% BSA to resuspend the cells, all processed samples were statically reacted at 4° C. for 30 minutes, then the cells were collected by centrifugation every 500 g for 3 minutes, washed with an equal volume of pre-cooled PBS containing 1% BSA and centrifuged 3 times, and then collected for later use.

### 1.4 Flow Detection:

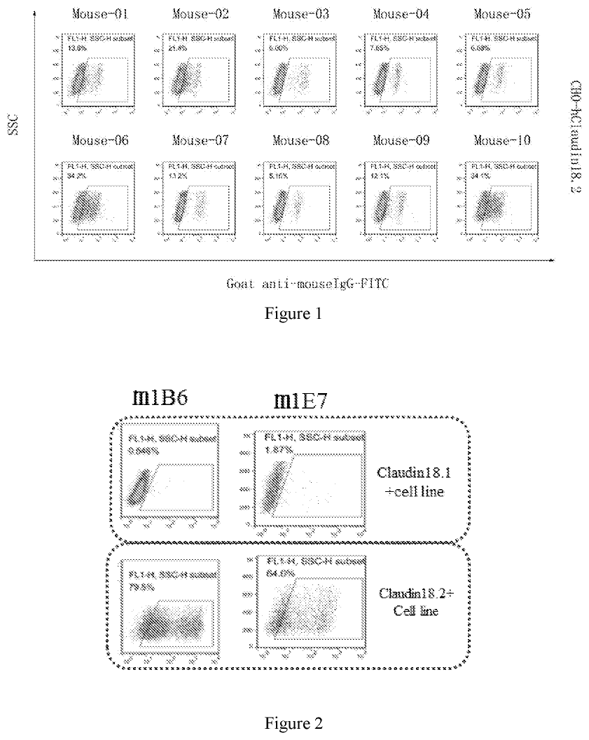

hClaudin18.2-CHO-NC and hClaudin18.1-CHO-NC samples were used to confirm the flow voltage. hClaudin18.2-CHO-mouse secondary antibody and hClaudin18.1-CHO-mouse secondary antibody samples were used to confirm the negative detection value. The established flow template to detect hClaudin18.2-CHO-m1B6 and hClaudin18.2-CHO-m1E7. The flow cytometry results of hClaudin18.1-CHO-m1B6 and hClaudin18.1-CHO-m1E7 samples showed that m1B6/m1E7 had a very good specific response, and the results were shown in FIG. 2.

### Example 2 The Affinity and Specificity Detection of Anti-Claudin 18.2 m1B6/m1E7 Monoclonal Antibody

Production and Purification of Anti-Claudin 18.2 m1B6/m1E7 Ascites

10 Female BA LB/c mice were taken and each was injected with 0.5 mL of paraffin oil into the abdominal cavity. The mice were ready for later use after 10 days. 10 Mice were divided into two cages, 5 in each cage. 1E+06 Cells of pre-treated 1B6/1E7 monoclonal cell line was injected into the abdominal cavity per mouse. After 10-12 days, the ascites produced by the mice was collected, and each cell line was collected about 10 mL of ascites for later use.

The collected ascites was centrifuged at 12000 g for 10 min to collect the supernatant, then 50% Saturated ammonium sulfate was added. The mixture was mixed thoroughly and stood for 30 min at 4° C., then centrifuged at 10000 g for 10 min to collect the precipitate. The precipitate was resuspended in an equal volume of PBS, filtered with a 0.45 μm filter membrane and ready for use.

PBS was used to equilibrate the protein A affinity chromatography column (5 mL pre-packed column) at a flow rate of 4 mL/min. After equilibrating 5 column volumes, the pre-processed m1B6/m1E7 was loaded and purified at a rate of 4 mL/min. After loading the sample, PBS was used to continue to rinse until the detection baseline was stable, then 0.1M pH3.5 acetic acid was used for elution. The elution peak was collected, and 1M tris buffer was used to adjust the pH of the eluate to pH7.4. After purification, the protein A chromatography column was washed with 0.1M NaOH buffer for 5CV, washed with PBS until the pH was neutral, then washed with purified water until the baseline of each test was stable. The protein A column was stored with 20% ethanol. The m1B6/m1E7 eluted sample was transferred to a 25 kD dialysis bag and dialyzed into PBS for later use.

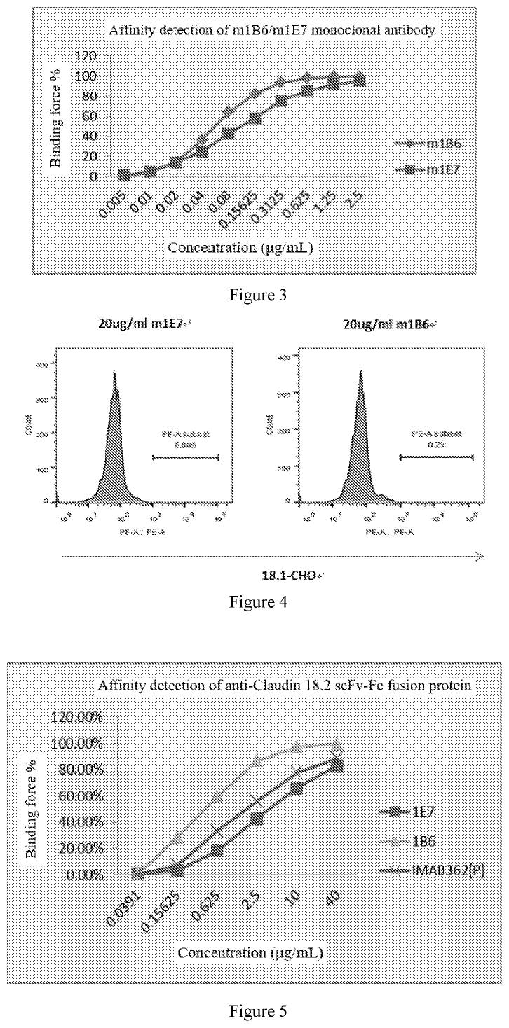

CHO overexpressing claudin18.1 cells and CHO overexpressing claudin18.2 cells were prepared, centrifuged at 300 g for 5 minutes, then resuspended in PBS, and this step was repeated twice. Finally, the concentration was adjusted to 3E+06 cells/mL with PBS. The 2 antibodies were diluted from 2.5 μg/mL in 2-fold gradient to 0.005 μg/mL with 10 gradients. CHO overexpressing claudin18.1 cells and CHO overexpressing claudin18.2 cells were laid out in two rows of transparent 96 round-bottomed wells, each with 100 μL, and then the antibodies were added to the cells in order and mixed evenly at 1:1. Each was set blank wells and negative wells. The mixture was put in a refrigerator at 4° C. and incubated for 1 hour. After the incubation, the mixture was centrifuged at 500 g for 3 min in a large-capacity benchtop high-speed centrifuge, then resuspended in PBS, and this step was repeated three times. The PE-labeled goat anti-mouse secondary antibody was diluted to a concentration of 1:500, and 100 μL was added to each well. The blank wells were left alone. The mixture was put in a refrigerator at 4° C. and incubated for 30 min. After the incubation, the mixture was centrifuged at 500 g for 3 min in a large-capacity tabletop high-speed centrifuge, then resuspended in PBS, and this step was repeated three times. Finally, 180 μL of PBS was added to each well and BDverse flow cytometer was used to test. The EC₅₀ of m1B6 was about 0.06 μg/mL, and the EC₅₀ of m1E7 was about 0.1 μg/mL. It did not cross-react with Claudin 18.1, and the concentration of 20 μg/mL did not cross-react with Claudin 18.1, so it had good specificity. The results were as shown in in FIG. 3 and FIG. 4.

### Example 3 The Activity Detection of Anti-Claudin 18.2 scfv-FC Fusion Protein

Beijing Qingke Biotechnology Co., Ltd. was entrusted to sequence 1B6 and 1E7 hybridoma cell lines. The expression vector of anti-claudin 18.2 scFv-Fc fusion protein was constructed by molecular cloning. The constructed expression vector was transiently transformed into 293F cells to express anti-Claudin 18.2 scFv-FC fusion protein. The medium supernatant was collected, centrifuged at 12000 g for 10 minutes and then ready for later use. Protein A chromatography column was used to equilibrate the protein A affinity chromatography column (5 mL pre-packed column) with PBS at a flow rate of 4 mL/min. After equilibrating 5 column volumes, the pre-processed m1B6/m1E7 (Fc fusion protein form) was loaded and purified at a rate of 4 mL/min. After loading the sample, PBS was used to continue to rinse until the detection baseline was stable, then 0.1M pH3.5 acetic acid was used for elution. The elution peak was collected, and 1M tris buffer was used to adjust the pH of the eluate to pH7.4. After purification, the protein A chromatography column was washed with 0.1M NaOH buffer for 5CV, washed with PBS until the pH was neutral, then washed with purified water until the baseline of each test was stable. The protein A column was stored with 20% ethanol. The 1B6/1E7 eluted sample was transferred to a 25 kD dialysis bag and dialyzed into PBS for later use.

CHO overexpressing claudin18.2 cells were prepared and centrifuged at 300 g for 5 minutes, then resuspended in PBS, and this step was repeated twice. Finally, the concentration was adjusted to 3E+06 cells/mL with PBS. The 3 antibodies m1B6/m1E7/IMAB362-FC were diluted from 40 μg/mL in 4-fold gradient to 0.04 μg/mL with 6 gradients. CHO overexpressing claudin18.1 cells and CHO overexpressing claudin18.2 cells were laid out in two rows of transparent 96 round-bottomed wells, each with 100 μL, and then the antibodies were added to the cells in order and mixed evenly at 1:1. Each was set blank wells and negative wells. The mixture was put in a refrigerator at 4° C. and incubated for 1 hour. After the incubation, the mixture was centrifuged at 500 g for 3 min in a large-capacity benchtop high-speed centrifuge, then resuspended in PBS, and this step was repeated three times. The PE-labeled goat anti-human secondary antibody was diluted to a concentration of 1:500, and 100 μL was added to each well. The blank wells were left alone. The mixture was put in a refrigerator at 4° C. and incubated for 30 min. After the incubation, the mixture was centrifuged at 500 g for 3 min in a large-capacity tabletop high-speed centrifuge, then resuspended in PBS, and this step was repeated three times. Finally, 180 μL of PBS was added to each well and BDverse flow cytometer was used to test. The EC₅₀ of m1B6-FC was about 0.5 μg/mL, the EC₅₀ of m1E7-FC was about 2.6 μg/mL, and the EC₅₀ of IMAB362-FC was about 2.0 μg/mL. m1B6 had a higher affinity, and m1E7 had the same affinity as existing clinical antibodies. The results were as shown in FIG. 5.

### Example 4 Anti-Claudin 18.2 Antibody 1B6/1E7 Epitope Identification

The different peptides of hClaudin 18.2 were synthesized according to the following amino acid sequence: 18.2EL1-A: DQWSTQDLYNNPVTAVFNYQGC, 18.2EL1-B: YQGLWRSCVRESSGFTECRG, 18.2EL1-C: CRGYFTLLGLPAmLQAVR, 18.2EL1-D: VRESSGFTECRGYFTLLGLP, 18.2EL1-E: DLYNNPVTAVFNYQGLWRSC, 18.2EL1-F: DQWSTQDLYNNPVTC, 18.2EL1-G: AVFNYQGLWRSC, 18.2EL1-H: CVRESSGFTE, 18.2EL1-I: CRGYFTLLGL. The nine synthetic peptides were dissolved with water:acetonitrile=3:1 ultrasonically, and the final concentration after dissolution was 1 mg/mL. Each peptide was divided into 100 μL/1.5 mL EP tubes for later use. m1B6 was diluted to 2 μg/mL for a total of 1 mL, m1E7 mAb was diluted to 4 μg/mL for a total of 1 mL, and IMAB362 was diluted to 20 μg/mL for a total of 1 mL. The diluted 3 anti-claudin18.2 antibodies were mixed well with the aliquoted peptides in a volume ratio of 1:1. The control group was set and the diluted m1B6, m1E7, IMAB362 were mixed well with PBS in a volume ratio of 1:1. The above-mentioned mixing system was put in a refrigerator at 4° C. for 30 minutes.

The CHO overexpressing claudin18.2 cells were collected, then centrifuged at 300 g for 5 min and resuspended with an equal volume of PBS. The step of centrifugation and resuspension was repeated twice. Finally, the concentration was adjusted to 3E+06 cells/mL with PBS. 100 μL of cell suspension (2 blank control wells and 1 negative control well) was added to each well of transparent 96 round-bottomed wells, and centrifuged at 300 g for 5 min to remove the supernatant and save the cell pellet for later use.

The incubated antibody peptide mixing system was added to the corresponding cell pellet, mixed well with the cells and labeled. Then the mixture was put in a refrigerator at 4° C. and incubated for 30 minutes. After the incubation is over, the mixture was centrifuged at 300 g for 5 minutes and resuspended in PBS. This step was repeated three times. The PE-labeled goat anti-mouse secondary antibody was diluted to a concentration of 1:500, and 100 μL was added to each well. The blank wells were left alone. The mixture was put in a refrigerator at 4° C. and incubated for 30 min. After the incubation, the mixture was centrifuged at 300 g for 5 min, then resuspended in PBS, and this step was repeated three times. Finally, 180 μL of PBS was added to each well and BD flow cytometer was used to detect the cell fluorescence intensity. m1B6 bound to a composite epitope composed of peptides A and E, wherein peptide A was its dominant epitope. m1E7 only bound to the epitope where A peptide was located. IMAB362 bound to a composite epitope composed of the A, C and E peptides, wherein the peptide E was its dominant epitope. The results were as shown in FIG. 6.

### Example 5 The Tumor Killing Activity Detection in Vitro of Anti-Claudin 18.2 CAR-T

Anti-Claudin18.2 CAR-T cell was constructed (m1B6/m1E7/IMAB362). The CAR structure was as described above, including: signal peptide-Anti-Claudin18.2 scfv-CD8 hinge+CD8TM-ICOS-4-1BB-CD3ζ, wherein the amino acid sequence of Anti-Claudin 18.2 scfv was sequence of 1B6 or 1E7, as shown in SEQ ID NO: 23 and SEQ ID NO: 24, respectively. The amino acid sequences of other structures (such as signal peptide, CD8 hinge, etc.) were shown in EQ ID NO: 25˜30.

293T cells were plated according to the amount of 6E+06 cells per 10 cm cell culture dish, and cultured overnight at 37° C. and 5% CO₂ for later use. Whether the plated cells reached 95%-99% confluence was observed the next day.

The lentivirus packaging system was prepared according to Table 1 (each was 10 cm packaging system, m1B6/m1E7/IMAB362/GFP lentivirus was prepared respectively).

TABLE 1

| Component | Volume | |
|---|---|---|
| A tube | | |
| Opti-MEM reduced serum medium | 1500 | μL |
| Lipofectamine 3000 transfection reagent | 41 | μL |
| B tube | | |
| Opti-MEM reduced serum medium | 1500 | μL |
| P3000 Enhancer reagent | 35 | μL |
| virapower lentivirus packaging mixture | 13 | μL |
| pLenti expression vector | 4.3 | μg |

After preparing A/B, the mixture of tube A was transferred to tube B. The resulting mixture was mixed gently and thoroughly, and then stood still for 10-20 minutes in the dark. 9 mL of DMEM medium containing 10% FBS was added and mixed thoroughly for later use.

The 293T cells (10 cm cell culture dish) was prepared in advance, and the medium supernatant was removed. The corresponding A/B mixed product was gently transferred to the corresponding cell culture dish, and the corresponding label was made. After culturing at 37° C. and 5% CO₂ for 6 hours, the mixture was replaced with fresh T cells containing healthy human T cells separated by Ficoll lymphocyte separation solution and cultured in a 24-well plate at 1E+06 cells per well. At the same time, CD3/CD28 antibody-coupled magnetic beads (Invitrogen) were added to stimulate T cells. After 48 hours, the corresponding lentivirus was added to infect. IL-2 (300 U/mL) was added during virus infection, and CAR-T cells were expanded to the 6th or 7th day to detect CAR gene expression and used in subsequent experiments.

The effective target ratio of CAR-T and effector cells (H460/18.2-H460) was set to 1:3, 1:1, 3:1, 9:1, and the effector cells were collected and centrifuged at 400 g for 5 min in a centrifuge tube. The supernatant was discarded. The resulting mixture was washed with an appropriate amount of PBS once, centrifuged and removed the supernatant, then 0.5 mL of CTS complete medium was added and the mixture was resuspended. The cell density and positive rate corresponding to CAR-T was detected. The cells were adjusted to a suitable density with CTS complete medium for later use. An appropriate amount of diluted effector cells was added to the 96-well cell detection plate, centrifuged at 400 g for 5 min to remove the supernatant. 100 μL of CAR-T of different densities was added to the corresponding wells, the cells were gently resuspended and mixed well, and then 100 μL of CTS was added to each well. The mixture was incubated for 20 hours, then test as follows:

After incubating for 20 hours, 20 μL of lysis buffer was added to the Vcc wells and TMR wells and mixed well. The mixture was lysed at 37° C. for 30 min.

The mixture was centrifuged at 400 g for 5 min at room temperature, and transferred 100 μL of the supernatant to a 96-well plate. The sample was also taken for cytokine release.

100 μL working solution was added to each well.

The mixture was incubated for 30 min in the dark at room temperature.

50 μL stop solution was added to each well and the absorbance at 490 nm was measured.

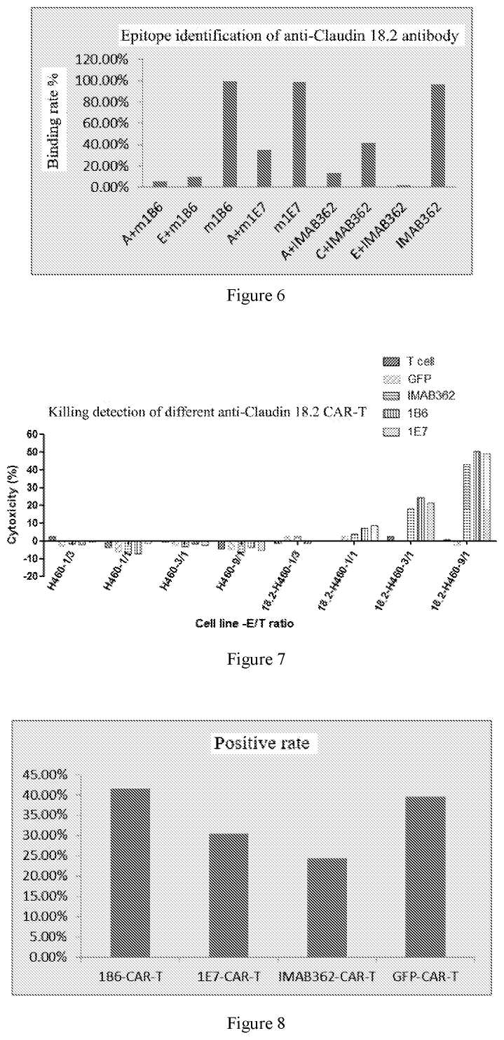

By comparison, it was found that the CAR-T constructed by 1B6/1E7 had obvious killing effect in vitro and had a significant dose-effect relationship. Compared with the CAR-T constructed by IMAB362, it had stronger killing effect and CAR positive rate. The results were as shown in in FIG. 7 and FIG. 8.

### Example 6 The Killing Activity Detection In Vivo of Anti-Claudin 18.2 CAR-T (Claudin 18.2 NCI-H460 Cells)

Claudin 18.2 NCI-H460 cells and NCI-H460 cells were cultured in RPMI-1640 medium (containing 10% FBS) and placed in a 37° C., 5% carbon dioxide incubator. When the cells grew to the required number, the cells were taken in the logarithmic growth phase. The original medium was discarded, and the mixture was trypsinized for 3 minutes. Then, the digestion was terminated with RPMI-1640 medium containing 10% FBS, the cells were collected and centrifuged at 1000 rpm for 5 min. After cell counting, the cell density was adjusted to 5E+07 cells/mL with a serum-free RPMI-1640 medium and Matrigel mixture (at a ratio of 1:1). Fixed NOD/SCID female mice were grabbed, and the cell suspension was injected into the right back of the mouse subcutaneously with 100 μL/mouse. In the Claudin 18.2 NCI-H460 model, animals were administered in groups when the tumor grew to about 150 mm³; in the NCI-H460 model, animals were administered in groups when the tumor grew to about 250 mm³. Each model was divided into Vehicle group, Anti-Claudin 18.2-1B6 CAR-T group, a total of 2 groups, each with 6 animals. Anti-Claudin18.2-1B6 CAR-T cells were collected and the cell density was adjusted to 1E+08 cells/mL with PBS solution. The cell suspension was injected into the tumor at 50 μL/mouse. In the Vehicle group, the tumor was injected with PBS solution at 50 μL/mouse. The tumor length and width were measured once every two days or twice a week, and the tumor volume was calculated (tumor volume=tumor length*tumor width*tumor width/2). The tumor growth inhibition rate (TGI) was calculated. If TX>T0, TGI=[1−TX/CX]*100%; if TX<T0, TGI=[1−(TX−T0)/T0]*100%. TX and CX were the tumor volume on the measurement day, T0 and C0 were the tumor volume on the day of administration. Statistical analysis was performed with SPSS16.0.

On the transplanted tumor model established by Claudin 18.2 NCI-H460 cells in NOD/SCID mice, Anti-Claudin 18.2-1B6 CAR-T could significantly inhibit tumor growth after intratumoral administration. At the end of the experiment, the tumor growth inhibition rate reached 134.78%. The tumor volume between the two groups was statistically analyzed, and there was a significant statistical difference P<0.01.

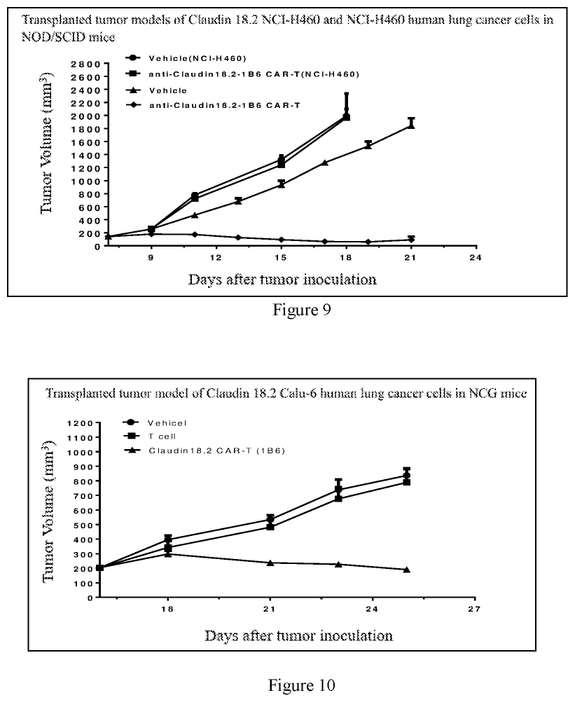

On the transplanted tumor model established by NCI-H460 cells in NOD/SCID mice, Anti-Claudin 18.2-1B6 CAR-T could not inhibit tumor growth after intratumoral administration. At the end of the experiment, the tumor growth inhibition rate was 2.17%. The tumor volume between the two groups was statistically analyzed, and there was no statistical difference. The results were as shown in FIG. 9.

### Example 7 The Killing Activity Detection In Vivo of Anti-Claudin 18.2 CAR-T (Claudin 18.2 Calu-6 Cells)

Claudin 18.2 Calu-6 cells were cultured in RPMI-1640 medium (containing 10% FBS) and placed in a 37° C., 5% carbon dioxide incubator. When the cells grew to the required number, the cells were taken in the logarithmic growth phase. The original medium was discarded, and the mixture was trypsinized for 3 minutes. Then, the digestion was terminated with RPMI-1640 medium containing 10% FBS, the cells were collected and centrifuged at 1000 rpm for 5 min. After cell counting, the cell density was adjusted to 2.5E+07 cells/mL with serum-free RPMI-1640 medium and Matrigel mixture (at a ratio of 1:1). Fixed NCG female mice were grabbed, and the cell suspension was injected into the right back of the mouse subcutaneously with 100 μL/mouse. When the tumor grew to about 150 mm³, the animals were administered in groups. The experiment was divided into Vehicle group, T cell group, Claudin 18.2 CAR-T (1B6) group, a total of 3 groups, each with 8 animals. Claudin 18.2 CAR-T (1B6) cells and T cells were collected and the cell density was adjusted to 1E+08 cells/mL with PBS solution. The cell suspension was injected into the tail vein at 200 μL/mouse. In the vehicle group, PBS solution was injected intravenously in the tail vein at 200 μL/mouse. The tumor length and width were measured twice a week, and the tumor volume was calculated (tumor volume=tumor length*tumor width*tumor width/2). The tumor growth inhibition rate (TGI) was calculated. If TX>T0, TGI=[1−TX/CX]*100%; if TX<T0, TGI=[1−(TX−T0)/T0]*100%. TX and CX were the tumor volume on the measurement day, T0 and C0 were the tumor volume on the day of administration. Statistical analysis was performed with SPSS16.0.

On the transplanted tumor model established by Claudin 18.2 Calu-6 cells in NCG mice, Claudin 18.2 CAR-T (1B6) could significantly inhibit tumor growth after intratumoral administration. At the end of the experiment, the tumor growth inhibition rate was 106.56%. The Claudin 18.2 CAR-T (1B6) group, Vehicle group and T cell group were statistically analyzed, and there was a significant statistical difference P<0.01. The results were as shown in FIG. 10.

### Example 8 The ADCC Activity Detection of Anti-Claudin 18.2 Chimeric Antibody

In this example, a Jurkat-NFAT-Luc-CD16 luciferase reporter gene cell line stably transfected with CD16 receptor and NFAT (Nuclear Factor of Activated T-cells) reaction element was used. When the test antibody (chimeric antibody 1B6, 1E7) and the Fab fragment of the control antibody IMAB362 bound to the antigen on the target cells BXPC-3-Claudin18.2, Capan-1-Claudin18.2, and SK-GT-Claudin18.2, the Fc segment of the antibody bound to the surface (Fc γRIIIA) of effector cells Jurkat-NFAT-Luciferase-CD16 cells, causing activation of NFAT-related signaling pathways in Jurkat-NFAT-Luciferase-CD16 cells, which in turn led to an increase in luciferase expression levels. The ADCC activity of Anti-Claudin 18.2 antibody was evaluated by detecting the luciferase expression level of effector cells Jurkat-NFAT-Luciferase-CD16 under the action of different concentrations (100 μg/mL, 20 μg/mL, 4 μg/mL, 0.8 μg/mL, 0.16 μg/mL, 0.032 μg/mL, 0.0064 μg/mL, 0.00128 μg/mL, 0.000256 μg/mL, 0.0000512 μg/mL) of the test antibody (chimeric antibody 1B6, 1E7) and the control antibody IMAB362. The results were as shown in FIG. 11. In the FIG. 11, the EC50 of the half-peak concentration reflected the ADCC activity of the antibody. The smaller the EC50 of the half-peak concentration, the stronger the ADCC activity of the antibody. The experimental results showed that on the target cell BXPC-3-Claudin 18.2, as the antibody concentration increased, the mean values of the test antibody (chimeric antibody 1B6, 1E7) and the control antibody IMAB362 gradually increased until reaching the plateau value, and half reached the peak concentration. The half-peak concentration EC50 is 0.002114 μg/mL, 0.002698 μg/mL and 0.003450 μg/mL, respectively; on the target cell Capan-1-Claudin 18.2, with the increase of the antibody concentration, the mean values of the test antibody (1B6, 1E7) and the control antibody IMAB362 gradually increased until reaching the plateau value, and the half-peak concentration EC50 is 0.002676 μg/mL, 0.002634 μg/mL and 0.003482 μg/mL, respectively; on the target cell SK-GT-Claudin 18.2, with the increase of the antibody concentration, the mean values of the test antibody (chimeric antibody 1B6, 1E7) and the control antibody IMAB362 gradually increased until reaching the plateau value, and the half-peak concentration EC50 is 0.004466 μg/mL, 0.007070 μg/mL and 0.009061 μg/mL, respectively; it could be seen that the ADCC activities of the tested antibodies 1B6 and 1E7 were better than the control antibody IMAB362.

### Example 9 The CDC Activity Detection of Anti-Claudin 18.2 Chimeric Antibody

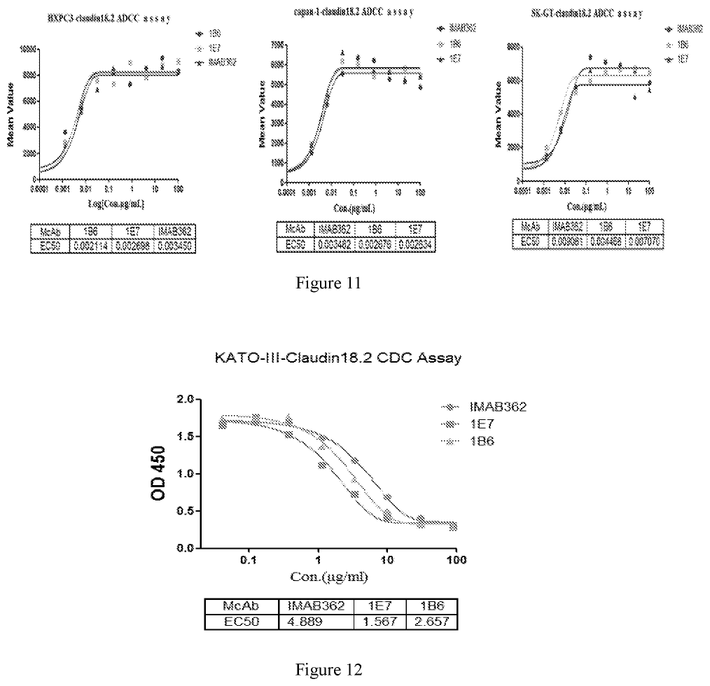

In this example, the CDC activity of Anti-Claudin 18.2 antibody was evaluated by detecting the cell viability of target cell KATOIII-3-Claudin 18.2 under the action of different concentrations (90 μg/mL, 30 μg/mL, 10 μg/mL, 3.33 μg/mL, 1.11 μg/mL, 0.37 μg/mL, 0.123 μg/mL, 0.041 μg/mL) of the test antibody (chimeric antibody 1B6, 1E7) and the control antibody IMAB362 by CCK8 method. The results were as shown in FIG. 12. In the FIG. 12, the OD450 value reflected the cell viability. The smaller the OD450 value, the worse the cell viability; the IC50 of the half inhibitory concentration reflected the CDC activity of the antibody. The smaller the IC50 of the half inhibitory concentration, the stronger the CDC activity of the antibody. The experimental results showed that with the increase of antibody concentration, the OD450 values of the test antibody (chimeric antibody 1B6, 1E7) and the control antibody IMAB362 gradually decreased until they approached zero, and the IC50 of the half inhibitory concentration was 2.656 μg/mL, 1.567 μg/mL and 4.889 μg/mL; it could be seen that the CDC activity of the tested antibodies 1B6 and 1E7 were better than the control antibody IMAB362.

### Example 10 The Anti-Tumor Efficacy Test of Anti-Claudin 18.2 Antibody Subcutaneous Xenograft Tumor

In order to evaluate the anti-tumor efficacy of Anti-Claudin 18.2 antibody in mice, the BXPC3˜18.2 subcutaneous xenograft tumor model was used to evaluate the anti-tumor efficacy of antibodies IE7 and 1B6. The human pancreatic cancer cells BXPC3˜18.2 in the logarithmic growth phase were taken and centrifuged. After the cells were counted, the cell density was adjusted to about 5.0*107/mL with serum-free RPMI-1640 medium and Matrigel mixture (at a ratio of 1:1). The volume of 0.1 mL/Mouse was injected subcutaneously into the back of nude mice. When the average tumor volume reached about 100 mm³, the drugs were administered in random groups. The mice were administered intravenously and intraperitoneally alternately. IMAB362, chimeric antibody 1E7, and 1B6 were administered at 10 mg/kg. Each mouse was administered 10 uL/g for 6 weeks, twice a week for the first three weeks, and once a week for the next three weeks. Starting from day 0 of administration, the size of the tumor and the weight of the mice were measured twice a week to calculate the trends of tumor volume and weight change. The tumor growth inhibition rate (TGI) was used as the test evaluation index. (TGI) %=[1−T/C]×100%, where T and C were the tumor volume at the end of the experiment. Statistical analysis was performed using SPSS16.0 software. One-way analysis of variance (one-way ANOVA) test was used for comparison between groups. P<0.05 (*) indicated statistical significance.

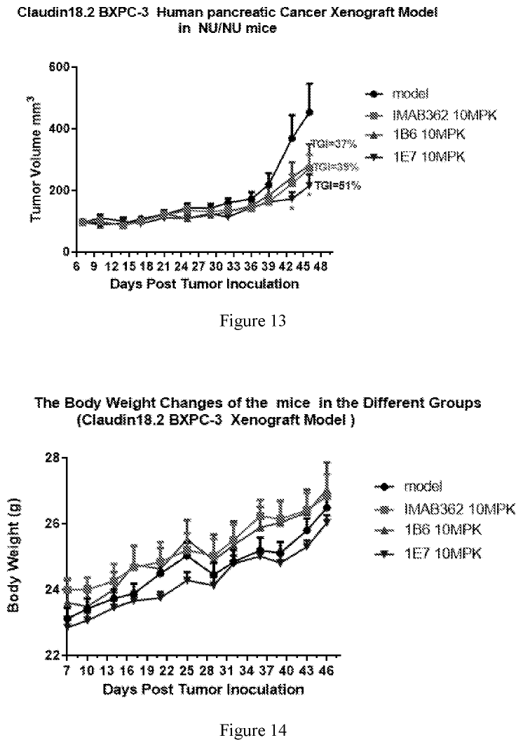

The results of the experiment were shown in FIGS. 13 and 14. Antibodies IMAB362, chimeric antibody 1E7, and 1B6 all had a certain inhibitory effect on the tumor volume of BXPC3˜18.2 subcutaneous xenograft tumor model. Antibodies IMAB362 and chimeric antibody 1B6 had the same inhibitory effect on tumor growth. TGI was 36˜39%. Chimeric antibody 1E7 had a better effect on inhibiting the growth of BXPC3˜18.2 tumors. TGI=51%. Compared with the control group, the difference was statistically significant; antibodies IMAB362 and 1B6, 1E7 had no effect on the weight gain of tumor-bearing mice.

Reference throughout this specification to “an embodiment”, “some embodiments”, “one embodiment”, “another example”, “an example”, “a specific example”, or “some examples” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. An antibody or antigen-binding fragment thereof having the ability of recognizing CLDN18.2, comprising:
a CDR sequence selected from at least one of the following or an amino acid sequence having at least 95% identity with it:
CDR sequences of light chain variable region shown in SEQ ID NO: 1, SEQ ID NO: 2, SEQ ID NO: 3, SEQ ID NO: 7, SEQ ID NO: 8 and SEQ ID NO: 9;
CDR sequences of heavy chain variable region shown in SEQ ID NO: 4, SEQ ID NO: 5, SEQ ID NO: 6, SEQ ID NO: 10, SEQ ID NO: 11 and SEQ ID NO: 12.

2. The antibody or antigen-binding fragment thereof according to claim 1, wherein the antibody comprises:
CDR1, CDR2, and CDR3 sequences of the light chain variable region shown in SEQ ID NO: 1, 2 and 3 respectively or the amino acid sequences having at least 95% identity with SEQ ID NO: 1, 2 and 3; or
CDR1, CDR2, and CDR3 sequences of the light chain variable region shown in SEQ ID NO: 7, 8 and 9 respectively or the amino acid sequences having at least 95% identity with SEQ ID NO: 7, 8 and 9; and/or
CDR1, CDR2, and CDR3 sequences of the heavy chain variable region shown in SEQ ID NO: 4, 5 and 6 respectively or the amino acid sequences having at least 95% identity with SEQ ID NO: 4, 5 and 6; or
CDR1, CDR2, and CDR3 sequences of the heavy chain variable region shown in SEQ ID NO: 10, 11 and 12 respectively or the amino acid sequences having at least 95% identity with SEQ ID NO: 10, 11 and 12.

3. (canceled)

4. The antibody or antigen-binding fragment thereof according to claim 1, wherein compared with peptide E, the antibody or antigen-binding fragment uses peptide A as the dominant epitope that specifically recognizes CLDN 18.2, the sequence of the peptide E having the amino acid sequence of SEQ ID NO:14, and the sequence of the peptide A having the amino acid sequence of SEQ ID NO:13.

5. The antibody or antigen-binding fragment thereof according to claim 1, comprising:
at least one of a heavy chain framework region sequence and a light chain framework region sequence,
wherein, at least a part of at least one of the heavy chain framework region sequence and the light chain framework region sequence is derived from at least one of a murine antibody, a human antibody, a primate antibody or a mutant thereof.

6. The antibody or antigen-binding fragment thereof according to claim 1, wherein the antibody has a light chain variable region with the amino acid sequence shown in SEQ ID NO: 15 or SEQ ID NO: 16,
and/or,
the antibody has a heavy chain variable region with the amino acid sequence shown in SEQ ID NO: 17 or SEQ ID NO: 18;
optionally, the antibody has a light chain variable region with the amino acid sequence shown in SEQ ID NO: 15 and a heavy chain variable region with the amino acid sequence shown in SEQ ID NO: 17;
optionally, the antibody has a light chain variable region with the amino acid sequence shown in SEQ ID NO: 16 and a heavy chain variable region with the amino acid sequence shown in SEQ ID NO: 18.

7. The antibody or antigen-binding fragment thereof according to claim 1, wherein the antibody comprises at least one of a heavy chain constant region and a light chain constant region, and at least a part of at least one of the heavy chain constant region and the light chain constant region is derived from at least one of a murine antibody, a human antibody, a primate antibody or a mutant thereof.

8. (canceled)

9. The antibody or antigen-binding fragment thereof according to claim 1, having
a light chain with the amino acid sequence shown in SEQ ID NO: 19, SEQ ID NO: 20, SEQ ID NO: 32 or SEQ ID NO: 34; and/or
a heavy chain with the amino acid sequence shown in SEQ ID NO: 21, SEQ ID NO: 22, SEQ ID NO: 31 or SEQ ID NO: 33.

10. (canceled)

11. The antibody or antigen-binding fragment thereof according to claim 1, wherein
the antibody is a single chain antibody, a chimeric antibody, a multimeric antibody, or a CDR-grafted antibody; or
the antigen-binding fragment comprises at least one of a Fab fragment, a (Fab)₂ fragment, a scFv-Fc fusion protein, a scFv-Fv fusion protein, an Fv fragment, and a minimum recognition unit.

12. The antibody or antigen-binding fragment thereof according to claim 11, wherein the antibody is a single chain antibody, and the single chain antibody has the amino acid sequence shown in SEQ ID NO: 23 or SEQ ID NO: 24.

13. (canceled)

14. A chimeric antigen receptor, comprising:
the extracellular region, wherein the extracellular region includes the heavy chain variable region and the light chain variable region of a single chain antibody, and wherein the light chain variable region and the heavy chain variable region are determined according to the antibody or antigen-binding fragment thereof as defined in claim 1.

15. The chimeric antigen receptor of claim 14, further comprising a transmembrane region and an intracellular region, wherein the transmembrane region includes the CD8 transmembrane region, and the intracellular region includes the intracellular segment of ICOS, 4-1BB and CD3ζ chain.

16. (canceled)

17. (canceled)

18. A nucleic acid molecule, wherein the nucleic acid molecule encodes the antibody or antigen-binding fragment thereof according to claim 1.

19. (canceled)

20. An expression vector, carrying the nucleic acid molecule of claim 18.

21. (canceled)

22. A recombinant cell, carrying the nucleic acid molecule of claim 18.

23. (canceled)

24. A pharmaceutical composition, comprising:
the antibody or antigen-binding fragment thereof according to
claim 1.

25. (canceled)

26. (canceled)

27. (canceled)

28. A method of diagnosing, treating or preventing CLDN 18.2 related diseases in a subject comprising administering to the subject a therapeutically effective amount of the antibody or antigen-binding fragment thereof of claim 1.

29. The method of claim 28, wherein the CLDN18.2 related disease includes tumors.

30. The method of claim 29, wherein the tumor comprises a solid tumor expressing CLDN 18.2, and optionally, the solid tumor comprises: gastric cancer, pancreatic cancer, esophageal cancer and lung cancer.

31. (canceled)

32. (canceled)

33. (canceled)

34. A kit for detecting CLDN 18.2, comprising the antibody or antigen-binding fragment thereof of claim 1.

35. (canceled)

36. A method of detecting CLDN 18.2 or diagnosing CLDN 18.2 related diseases in a subject using a kit comprising the antibody or antigen-binding fragment thereof of claim 1.

37. (canceled)

38. (canceled)

39. (canceled)

40. (canceled)


SEQUENCE LISTING (34 sequences)

SEQ ID NO: 1 - protein, 14 aa, Artificial Sequence
  CDR sequence of light chain variable region
SQSLLNSGNQKNYL

SEQ ID NO: 2 - protein, 5 aa, Artificial Sequence
  CDR sequence of light chain variable region
YWAST

SEQ ID NO: 3 - protein, 11 aa, Artificial Sequence
  CDR sequence of light chain variable region
CQNDYSYPFTF

SEQ ID NO: 4 - protein, 10 aa, Artificial Sequence
  CDR sequence of heavy chain variable region
SGYTFTSYWM

SEQ ID NO: 5 - protein, 10 aa, Artificial Sequence
  CDR sequence of heavy chain variable region
MIHPNSGSTN

SEQ ID NO: 6 - protein, 14 aa, Artificial Sequence
  CDR sequence of heavy chain variable region
CARRYYGSISPDYW

SEQ ID NO: 7 - protein, 14 aa, Artificial Sequence
  CDR sequence of light chain variable region
SQSLFNSGNQKNYL

SEQ ID NO: 8 - protein, 5 aa, Artificial Sequence
  CDR sequence of light chain variable region
YWAST

SEQ ID NO: 9 - protein, 11 aa, Artificial Sequence
  CDR sequence of light chain variable region
CQNDYSYPLTF

SEQ ID NO: 10 - protein, 10 aa, Artificial Sequence
  CDR sequence of heavy chain variable region
SGYTFTDYNM

SEQ ID NO: 11 - protein, 10 aa, Artificial Sequence
  CDR sequence of heavy chain variable region
YINPNNGGTS

SEQ ID NO: 12 - protein, 10 aa, Artificial Sequence
  CDR sequence of heavy chain variable region
CVTTRYLAVW

SEQ ID NO: 13 - protein, 22 aa, Artificial Sequence
  Peptide A
DQWSTQDLYNNPVTAVFNYQGC

SEQ ID NO: 14 - protein, 20 aa, Artificial Sequence
  Peptide E
DLYNNPVTAVFNYQGLWRSC

SEQ ID NO: 15 - protein, 113 aa, Artificial Sequence
  Light chain variable region
DIVMTQSPSSLSVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 16 - protein, 113 aa, Artificial Sequence
  Light chain variable region
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYFCQNDYSYPLTFGAGTKLELR

SEQ ID NO: 17 - protein, 119 aa, Artificial Sequence
  Heavy chain variable region
QVQLQQPGSELVKPGASVKLSCKASGYTFTSYWMHWVKQRPGQGLEWIGMIHPNSGSTNYNEKFKSKATLTVDKSSSTAYMQLSSLTSEDSAVYYCARRYYGSISPDYWGQGTTLTVSS

SEQ ID NO: 18 - protein, 115 aa, Artificial Sequence
  Heavy chain variable region
EVQLQQSGPELVRPGASVKMSCKASGYTFTDYNMHWVKQSHGKSLEWIGYINPNNGGTSYNQKFKGKATLTVNKSSSTAYMELRSLTSEDSAVYYCVTTRYLAVWGTGTTVTVSS

SEQ ID NO: 19 - protein, 220 aa, Artificial Sequence
  Light chain
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYFCQNDYSYPLTFGAGTKLELRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 220 aa, Artificial Sequence
  Light chain
DIVMTQSPSSLSVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 451 aa, Artificial Sequence
  Heavy chain
EVQLQQSGPELVRPGASVKMSCKASGYTFTDYNMHWVKQSHGKSLEWIGYINPNNGGTSYNQKFKGKATLTVNKSSSTAYMELRSLTSEDSAVYYCVTTRYLAVWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 22 - protein, 455 aa, Artificial Sequence
  Heavy chain
QVQLQQPGSELVKPGASVKLSCKASGYTFTSYWMHWVKQRPGQGLEWIGMIHPNSGSTNYNEKFKSKATLTVDKSSSTAYMQLSSLTSEDSAVYYCARRYYGSISPDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 23 - protein, 243 aa, Artificial Sequence
  Sequence of single chain antibody
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYFCQNDYSYPLTFGAGTKLELRGGGGSGGGGSGGGGSEVQLQQSGPELVRPGASVKMSCKASGYTFTDYNMHWVKQSHGKSLEWIGYINPNNGGTSYNQKFKGKATLTVNKSSSTAYMELRSLTSEDSAVYYCVTTRYLAVWGTGTTVTVSS

SEQ ID NO: 24 - protein, 247 aa, Artificial Sequence
  Sequence of single chain antibody
DIVMTQSPSSLSVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGSELVKPGASVKLSCKASGYTFTSYWMHWVKQRPGQGLEWIGMIHPNSGSTNYNEKFKSKATLTVDKSSSTAYMQLSSLTSEDSAVYYCARRYYGSISPDYWGQGTTLTVSS

SEQ ID NO: 25 - protein, 17 aa, Artificial Sequence
  Amino acid sequence of signal peptide of chimeric antigen receptor
MGVKVLFALICIAVAEA

SEQ ID NO: 26 - protein, 45 aa, Artificial Sequence
  Amino acid sequence of CD8 hinge of chimeric antigen receptor
TTTPAPRPPTPAPTIASQPLSLRPEACRPAAGGAVHTRGLDFACD

SEQ ID NO: 27 - protein, 24 aa, Artificial Sequence
  Amino acid sequence of CD8TM of chimeric antigen receptor
IYIWAPLAGTCGVLLLSLVITLYC

SEQ ID NO: 28 - protein, 38 aa, Artificial Sequence
  Amino acid sequence of ICOS of chimeric antigen receptor
CWLTKKKYSSSVHDPNGEYMFMRAVNTAKKSRLTDVTL

SEQ ID NO: 29 - protein, 42 aa, Artificial Sequence
  Amino acid sequence of 4-1BB of chimeric antigen receptor
KRGRKKLLYIFKQPFMRPVQTTQEEDGCSCRFPEEEEGGCEL

SEQ ID NO: 30 - protein, 113 aa, Artificial Sequence
  Amino acid sequence of CD3 zeta of chimeric antigen receptor
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 445 aa, Artificial Sequence
  Heavy chain of chimeric antibody IB6
EVQLQQSGPELVRPGASVKMSCKASGYTFTDYNMHWVKQSHGKSLEWIGYINPNNGGTSYNQKFKGKATLTVNKSSSTAYMELRSLTSEDSAVYYCVTTRYLAVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 220 aa, Artificial Sequence
  Light chain of chimeric antibody IB6
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYFCQNDYSYPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 449 aa, Artificial Sequence
  Heavy chain of chimeric antibody IE7
QVQLQQPGSELVKPGASVKLSCKASGYTFTSYWMHWVKQRPGQGLEWIGMIHPNSGSTNYNEKFKSKATLTVDKSSSTAYMQLSSLTSEDSAVYYCARRYYGSISPDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Artificial Sequence
  Light chain of chimeric antibody IE7
DIVMTQSPSSLSVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC